(19) United States
(12) Patent Application Publication
(10) Pub. No.: US 2006/0111335 A1
(43) Pub. Date: May 25, 2006

(54) Photoactivated anti-viral and anti-cancer agent

(76) Inventors: Harry Morrison, West Lafayette, IN (US); Elton Menon, Mason, OH (US); Devanesan Loganathan, West Lafayette, IN (US); Maribel Coromoto Navarro Acosta, Miranda (VE); Mark A. Billadeau, Knoxville, MD (US)

Correspondence Address: MUETING, RAASCH & GEBHARDT, P.A., P.O. BOX 581415, MINNEAPOLIS, MN, 55458, US

(21) Appl. No.: 10/523,315

(22) PCT Filed: Aug. 1, 2003
(86) PCT No.: PCT/US03/24096

§ 371 Date: Sep. 16, 2005

Related U.S. Application Data
(60) Provisional application No. 60/400,262, filed on Aug. 1, 2002.

(51) Int. Cl. A61K 31/555; C07F 15/00

(52) U.S. Cl. 514/185; 546/2

(57) ABSTRACT

Bisbipyridyl rhodium (III) compound and methods for photo-induced inactivation of pathogenic contaminants in biological materials.

This application claims the benefit of U.S. Provisional Application Ser. No. 60/400,262, filed Aug. 1, 2002, which is incorporated herein by reference in its entirety.

### STATEMENT OF GOVERNMENT RIGHTS

This invention was made with government support under a grant from the Public Health Service, National Institutes of Health, Grant No. GM56279. The U.S. Government has certain rights in this invention.

### BACKGROUND OF THE INVENTION

The use of light activated drugs is a very attractive option for the development of therapeutic methodologies that minimize damage to normal tissue by allowing spatial and temporal control over toxicity. Light activated drugs have been used for blood sterilization. For example, U.S. Pat. No. 6,030,767 (Wagner et al.) describes photosterilizing blood and blood components, such as red blood cells or plasma, using methylene blue derivatives. See also, e.g., U.S. Pat. No. 5,545,516 (Wagner) and U.S. Pat. Publ. 2001/0046662 A1 (Wagner et al.). Photodynamic therapy (PDT) has also been used as a treatment for several forms of cancer. PDT works by exposing a photosensitizing drug to specific wavelengths of light in the presence of oxygen. When this reaction occurs, the normally innocuous photosensitizing drug becomes cytotoxic via an activated species of oxygen, known as singlet oxygen. Some of these phototoxic agents can be given orally and are preferentially retained by tumor cells PDT has been used effective for ovarian cancer, lung cancer, breast cancer, esophogeal cancer, skin cancers and bladder cancers. However, because the effects of almost all PDT drugs are oxygen dependent, photosensitization typically does not occur in anoxic areas of tissue. For example, in vivo studies showed that induction of tissue hypoxia, by clamping, abolished the PDT effects of porphyrins (Gomer et al., Photochem. Photobiol., 40:435-439 (1984)).

These methodologies, including those blood decontamination procedures that employ methylene blue or its derivatives, rely heavily on the efficacy of the photosensitized formation of reactive oxygen species (ROS) by the light-absorbing agent. Reactive oxygen species include singlet (excited state) oxygen, hydroxy radical and superoxide anion. The concomitant need to overcome tumor cell resistance due to hypoxia, and to avoid the indiscriminate damage to biological blood components by ROS, makes the development of photoactivated agents that utilize oxygen-independent mechanisms for toxicity a worthy goal.

In order for a viral inactivation strategy to be effective for cellular blood components, it must selectively target viral agents and not inflict irreparable damage to biological components of the blood. The specific targeting of DNA and/or RNA is especially attractive for blood photosterilization since blood platelets, erythrocytes and plasma proteins do not contain genomic nucleic acid. Although white blood cells do contain nucleic acids, blood and blood products are typically partially or wholly leukodepleted prior to administration to patients. Moreover, white blood cells are often infected by the pathogen, so nonspecific killing of white blood cells is not always undesirable.

The recent observations that infectious agents such as West Nile virus, or agents currently unidentified, may adventitiously enter the blood supply and cause unintended disease in transfusion recipients argue for a prophylactic treatment of blood products. In addition, the heightened concerns regarding bioterrorism suggest that new precautions might be necessary to screen against a wide variety of viruses.

Recently, there has been increasing interest in the potential use of photoactivated organometallic complexes as a source of nucleic acid inactivation (Erkkila et al., Chem. Rev., 99:2777-2795 (1999); Billadeau et al., Metal Ions Biol. Syst., 33:269-296 (1996); Ali et al., Chem. Rev. 99:2379-2450 (1999)). When irradiated with UV light a number of such complexes exhibit powerful nuclease activity and/or the capability to form covalent bonds with DNA and RNA bases. One such chemical family includes bisbipyridyl rhodium (III) complexes, an example of which is cis-dichlorobis(1,10-phenanathroline) rhodium(III) chloride (also known as cis-Rh(phen)₂Cl₂⁺, or simply BISPHEN) (FIG. 1a). BISPHEN is a soluble metal complex, consisting of rhodium metal, two bidentated planar aromatic ligands (phen ligands, FIG. 2a), and two chloride ligands in a cis arrangement. The complex belongs to a relatively well-studied group of d⁶ metal complexes that are thermally stable and readily photoaquates when irradiated by a UVA light source. The photochemistry of BISPHEN involves ligand substitution from an excited triplet state (Crosby et al., J. Phys. Chem., 80:2206-2211 (1976)) with photoaquation occurring via a dissociative mechanism (Muir et al., Inorg. Chem., 12:1831-1835 (1973); Vanquickenborne et al., Inorg Chem., 17:2730-2736 (1978)). Upon irradiation, this complex covalently binds to nucleic acid, primarily to guanosine (Mahnken et al., Photochem. Photobiol., 49:519-520 (1989); Mahnken et al., J. Am. Chem. Soc., 114:9253-9265 (1992); Billadeau et al., Inorg. Chem., 33:5780-5784 (1994); Harmon et al., Inorg. Chem., 34:4937-4938 (1995); Mohammad et al., Biorg. Med. Chem. Lett., 9:1703-1708 (1999); Morrison et al., Photochem. Photobiol., 72:731-738 (2000)), crosslinks RNA (Mohammad et al., Biorg. Med. Chem. Lett., 9:1703-1708 (1999)), and inactivates naked viral DNA (Mohammad et al., Photochem Photobiol., 59:189-196 (1994)).

BISPHEN is phototoxic to both single stranded (ss) and double stranded (ds) naked infectious DNA from phages S13 and G4, with covalent binding of the complex to nucleic acid playing a major role in the toxicity towards ss DNA. (Mohammad et al., Photochem. Photobiol., 59:189-196 (1994)). Most recently, BISPHEN has been shown to crosslink packaging RNA upon irradiation (Mohammad et al., Biorg. Med. Chem. Lett., 9:1703-1708 (1999)). This is only the second class of molecules known to cross-link nucleic acid upon irradiation, and represents the first example of the photoinitiated crosslinking of purines (Rajiski et al., Chem. Rev., 28:2723-2795 (1998)).

Even though BISPHEN appeared to have potential as a therapeutic photoactivated metal complex, it has significant limitations. It lacks the hydrophobicity needed for uptake by mammalian cells. For example, it is not taken up by HeLa (human cervical carcinoma) or KB (human nasopharingeal carcinoma) cells (Terrian, “The photochemistry and photobiology of rhodium (III) polypyridyl complexes and psoralen pro-drugs,” PhD dissertation, Purdue University, West Lafayette, Ind. (1996)). Membrane permeability is important in order to produce bioactivity against intracellular pathogens. In addition, although it is active against naked viral DNA, it showed minimal phototoxicity against Sindbis virus, which is a more complex viral system (Houghtaling, “Photochemical and photobiological properties of phenothiazine dyes and an analogue of urocanic acid as potential anticancer and virucidal agents,” PhD dissertation, Purdue University, West Lafayette, Ind. (1998)), and it did not exhibit any activity against bacteriophage φ6 (Terrian, “The photochemistry and photobiology of rhodium (III) polypyridyl complexes and psoralen pro-drugs,” PhD dissertation, Purdue University, West Lafayette, Ind. (1996)). Another shortcoming of BISPHEN is that it exhibits minimal dark association with DNA (Mahnken et al., J. Am. Chem. Soc., 114:9253-9265 (1992)), likely due to the absence of a ligand capable of intercalation. BISPHEN exhibited very low association levels with DNA, and only when the ionic strength of the solution was well below that of blood sera, thus raising concerns about the ultimate selectivity of the molecule for cellular and viral nucleic acid. A possible indication that the lack of selectivity for DNA plays a role in BISPHEN's low bioactivity is the fact that, even when delivered to cells by liposome, the complex exhibited marginal levels of phototoxicity against KB cells (Terrian, “The photochemistry and photobiology of rhodium (III) polypyridyl complexes and psoralen pro-drugs,” PhD dissertation, Purdue University, West Lafayette, Ind. (1996)). Light penetrates deeper into tissues as its wavelength increases, and it is desirable that a photoactivated compound be biologically active at longer wavelengths.

In an attempt to address the shortcomings of BISPHEN, cis-dichloro(1,10-phenanthroline)(9,10-phenathrenequinone diimine)rhodium(III) chloride (cis-Rh(phen)(phen)Cl₂⁺, or PHENPHI) (FIG. 1b), was synthesized (Terrian, “The photochemistry and photobiology of rhodium (III) polypyridyl complexes and psoralen pro-drugs,” PhD dissertation, Purdue University, West Lafayette, Ind. (1996)). In this complex, one of the 1,10-phenanthroline (phen) ligands has been replaced by 9,10-phenanthrenequinone diimine (phi) ligand (FIG. 2d). PHENPHI has been shown to associate in the dark with DNA with a K_{eq} of 3.7×10⁴ M⁻¹ (Terrian, “The photochemistry and photobiology of rhodium (III) polypyridyl complexes and psoralen pro-drugs,” PhD dissertation, Purdue University, West Lafayette, Ind. (1996)). As in the case of BISPHEN, the complex covalently binds to DNA when irradiated with light (Terrian, “The photochemistry and photobiology of rhodium (III) polypyridyl complexes and psoralen pro-drugs,” PhD dissertation, Purdue University, West Lafayette, Ind. (1996)). In addition, PHENPHI has exhibited photonuclease activity (Mohammad et al., Photochem. Photobiol., 67:95S (1998)). PHENPHI does not require liposomes to penetrate the cell membrane, and produces substantial photoinduced cell death. It also shows potent virucidal activity against bacteriophage φ6 and Sindbis virus (Terrian, “The photochemistry and photobiology of rhodium (I polypyridyl complexes and psoralen pro-drugs,” PhD dissertation, Purdue University, West Lafayette, Ind. (1996); Houghtaling, “Photochemical and photobiological properties of phenothiazine dyes and an analogue of urocanic acid as potential anticancer and virucidal agents,” PhD dissertation, Purdue University, West Lafayette, Ind. (1998)).

However PHENPHI also has less desirable traits. It is thermodynamically less stable than BISPHEN, decomposing at room temperature when in Tris buffer pH 7, and at 37° C. when in phosphate-buffered saline (PBS) pH 7. It readily substitutes acetate for chloride ligand in acetate buffer pH 5. Though its added hydrophobicity allows for cell membrane transport, it shows substantial binding affinity for protein, which can be a problem when the sample being treated, such as plasma, contains a significant amount of extracellular protein. This is manifested by the ability of plasma protein to inhibit PHENPHI's ability to kill virus in blood samples (Terrian, “The photochemistry and photobiology of rhodium (III) polypyridyl complexes and psoralen pro-drugs,” PhD dissertation, Purdue University, West Lafayette, Ind. (1996); Terrian, Photochem. Photobiol., 65:68S (1997)), and in the inhibitory effect of added fetal calf serum on the complex's phototoxicity towards Sindbis virus (Houghtaling, “Photochemical and photobiological properties of phenothiazine dyes and an analogue of urocanic acid as potential anticancer and virucidal agents,” PhD dissertation, Purdue University, West Lafayette, Ind. (1998)).

The development of therapeutic agents that retain the toxicity of BISPHEN but are stable, optimized for hydrophobicity, and readily intercalate nucleic acids would represent a significant advance in the art.

### SUMMARY OF THE INVENTION

The invention provides a bisbipyridyl rhodium (III) compound, together with methods of using the compound to inactivate pathogenic contaminants in biological materials and to treat various human and animal diseases. In one embodiment, the bisbipyridyl rhodium (III) compound has the following formula:

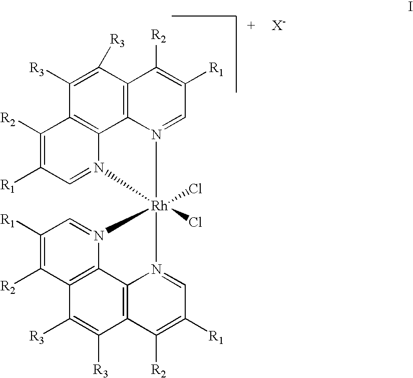

In another embodiment, the bisbipyridyl rhodium (III) compound has the following formula: 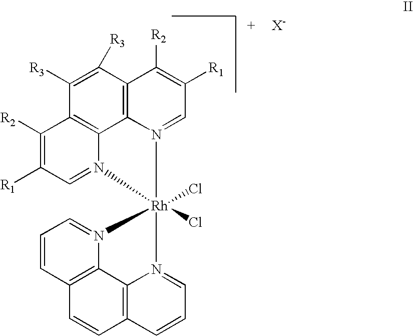
wherein R₁, R₂ and R₃ are each independently selected from the group consisting of an alkyl group, an alkenyl group, an alkynyl group, a nitrile, an azide, an aryl group, an aralkyl group, a heteroaryl group, a hydroxy group, an alkoxy group, an aryloxy group, an amine group, and a hydrogen atom, or any two of R₁, R₂ and R₃ together form an aryl or heteroaryl ring, and wherein X is a counterion. Optionally, where R₁=R₃=H, R₂ is neither methyl nor phenyl.

In another embodiment, the bisbipyridyl rhodium (III) compound has the following formula: 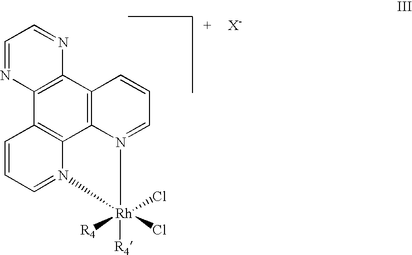
wherein R₄ and R₄′ together form a phen ligand, yielding cis-dichloro{2,3-di(2-pyridyl)quinoxaline}{1,10-phenanthroline}rhodium (III)chloride (TAPPHEN) or a “tap” ligand, yielding cis-dichlorobis{2,3-di(2-pyridyl)quinoxaline} rhodium (III) chloride (BISTAP, FIG. 1k).

In yet another embodiment, the bisbipyridyl rhodium (III) compound has the following formula: 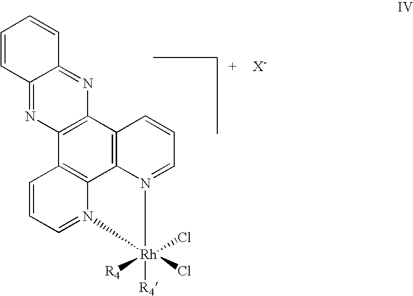
wherein R₄ and R₄′ together form a phen ligand, yielding cis-dichloro(dipyrido[3,2a-2′3′c]phenazine)(1,10-phenantroline) rhodium(III) chloride (DPPZPHEN) (FIG. 1c) or a dppz ligand (FIG. 2b), yielding cis-dichlorobis{dipyrido(3,2-a: 2′,3′-c)phenazine}rhodium (III) chloride (BISDPPZ) (FIG. 11).

Pathogenic contaminants in a biological material can be reduced by contacting the sample with any of the bisbipyridyl rhodium (III) compounds described herein, then irradiating the biological material for a time sufficient to activate the bisbipyridyl rhodium (III) compound thereby causing a reduction the level of pathogenic contaminants in the biological material. The pathogenic contaminant can be a pathogenic organism such as a bacterium, virus and protozoan, or it can be a cell from the patient, such as a leukocyte or a tumor cell. The method of claim 17 wherein the pathogenic contaminant comprises a tumor cell. The biological material is typically irradiated at with light having a wavelength of 310 nm to 400 nm, preferably 320 nm to 400 nm, although light having a wavelength over 400 nm will also cause photoactivation of the compound.

The decontamination method is well-suited for the sterilization of blood or blood components, particularly blood products that are substantially free of hemoglobin, including platelets, concentrated platelets, plasma, serum and blood protein fractions. Optionally, the bisbipyridyl rhodium (III) compound is removed from the biological material after photoactivation. The method can also be used to treat infectious or somatic disease in a patient. For example, it can be used to treat viral or bacterial infections, or to kill disease cells in a patient, such as tumor cells.

Optionally, the method includes, prior to photoactivation, contacting the biological material with a sensitizer molecule having an absorption maximum of greater than 550 nm. The biological material is then irradiated with light having a wavelength of greater than 550 nm so as to excite the sensitizer molecule and thereby indirectly activate the bisbipyridyl rhodium (III) complex. Because it utilizes light having longer wavelengths, this embodiment of the method advantageously allows decontamination of biological materials such as blood or blood components that contain hemoglobin. Representative sensitizer molecules include dyes such as methylene and acridine orange.

Preferred bisbipyridyl rhodium (III) compounds for use in the method of the invention include cis-dichloro(dipyrido[3,2a-2′3′c]phenazine)(1,10-phenantroline) rhodium(III) chloride (DPPZPHEN), cis-dichlorobis(3,4,7,8-tetramethyl-1,10-phenanthroline) rhodium(III) chloride (OCTMP), cis-dichlorobis{dipyrido(3,2-a: 2′,3′-c)phenazine}rhodium (III) chloride (BISDPPZ), cis-dichlorobis(3,7-dimethoxy-1,10-phenanthroline) rhodium(III) chloride (TMOBP), cis-dichlorobis(3,7-diisopropoxy-1,10-phenanthroline) rhodium(III) chloride (TIOBP), cis-dichlorobis{3,7(N,N-dimethylamino)-1,10-phenanthroline} rhodium(III) chloride (BISNMe2), cis-dichlorobis(4,7-diphenyl-1,10-phenanthroline) rhodium(III) chloride (TPBP), and cis-dichlorobis{2,3-di(2-pyridyl)quinoxaline} rhodium (III) chloride (BISTAP).

### BRIEF DESCRIPTION OF THE DRAWINGS

FIG. 1 shows structures for a) BISPHEN, b) PHENPHI, c) DPPZPHEN, d) 37TMBP, e) 56TMBP, f) OCTBP, g) BISNMe₂, h) TMOBP, i) TIOBP, j) TPBP, k) BISTAP, and l) BISDPPZ; as well as m) UV-Vis molar absorption spectra for BISPHEN and DPPZPHEN.

FIG. 2 shows representative ligands used to form rhodium (III) complexes: a) phen; b) dppz; c) tap; and d) phi.

FIG. 3 shows histograms of the size exclusion chromatography of nucleic acid product from the irradiation of DPPZPHEN with CT DNA. Histogram A: UV-Vis absorption analysis of the samples at 260 nm; Histogram B: UV-Vis absorption analysis of the samples at 380 nm.

FIG. 4 shows a) light dose dependency of photonicking of φX-174 plasmid DNA by DPPZPHEN with 311 nm irradiation. Lanes: 1, Plasmid with DPPZPHEN in the dark; 2, irradiation for 5 min; 3, irradiation for 10 min; 4, irradiation for 15 minutes; and b) irradiation of DPPZPHEN with φX-174 plasmid DNA for 10 minutes in presence of ROS quenchers. Lanes: 1, DPPZPHEN in the dark; 2, DPPZPHEN with 25 μg/mL SOD; 3, DPPZPHEN with 8 mM histidine; 4, DPPZPHEN 12 μM with 6 mM mannitol; 5, DPPZPHEN, no quenchers. RFI is the circular supercoiled plasmid DNA form, and RF II is the circular relaxed plasmid DNA form.

FIG. 5 shows irradiation of DPPZPHEN with tumor cell lines with 311 nm light. Series: (□) irradiation of DPPZPHEN with GN4 cells, (Δ) irradiation of DPPZPHEN with M109 cells, and (▪) irradiation of DPPZPHEN with KB cells. Controls: (X) KB cells irradiated without DPPZPHEN, (●) M109 cells irradiated without DPPZPHEN and (O) GN4 cells irradiated without DPPZPHEN. The time=0 points correspond to samples from series (Δ), (□), and (▪) that were incubated with DPPZPHEN in the dark.

FIG. 6 shows a) Series (□) is the irradiation of 45 μM DPPZPHEN with SINV, and series (Δ) is the control irradiation of SINV without DPPZPHEN, both using the 355 nm laser. Zero absorbed dose data point series (O) represents SINV incubated with DPPZPHEN in the dark; and b) Simple hit theory analysis of data in series (♦). Simple hit theory states that one hit is responsible for inactivation of one viral particle according to the formula S=e^{-kD}, where S is the surviving fraction of virus and D is the absorbed dose in photons (Hiatt, Bacteriol. Rev., 28:150-163 (1964)).

FIG. 7 shows OCTBP uptake by KB cells.

FIG. 8 shows MTT assay of KB cell survival after OCTBP and irradiation at 311 nm, 70 μM.

FIG. 9 shows MTT assay of GN4 cell survival after OCTBP and irradiation at 311 nm a) 55 μM and b) 75 μM.

FIG. 10 shows MIT assay of M109 cell survival after OCTBP and irradiation at 311 nm, 55 μM.

FIG. 11 shows MTT assay of KB cell survival after OCTBP and irradiation at >400 nm a) 55 μM and b) 63 μM.

### DETAILED DESCRIPTION OF THE ILLUSTRATIVE EMBODIMENTS

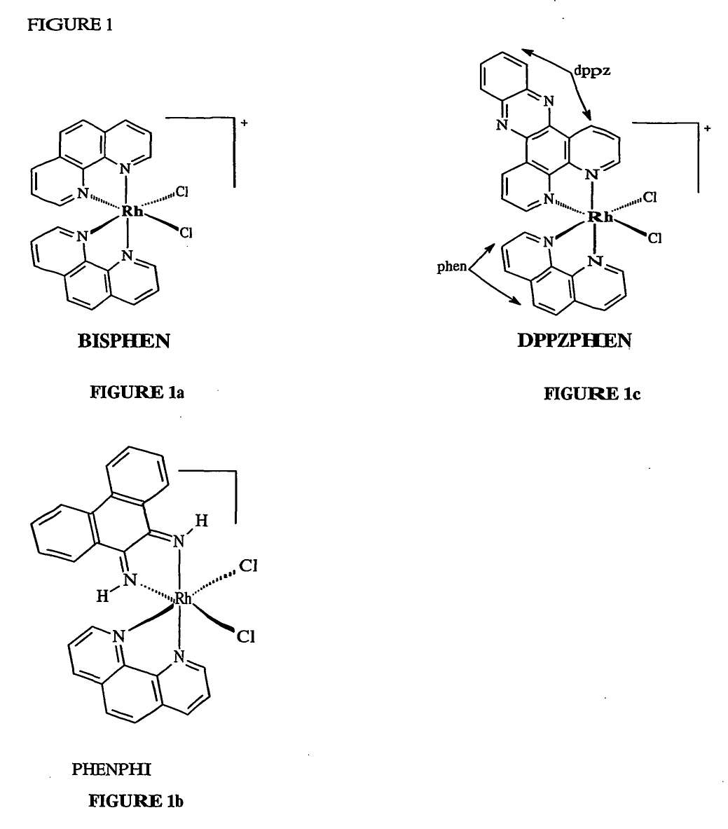

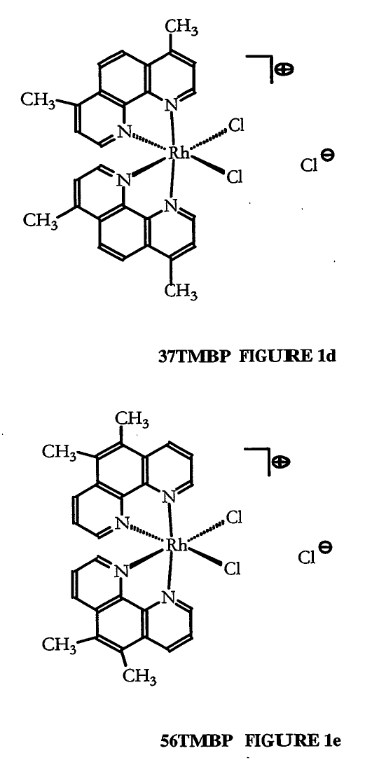

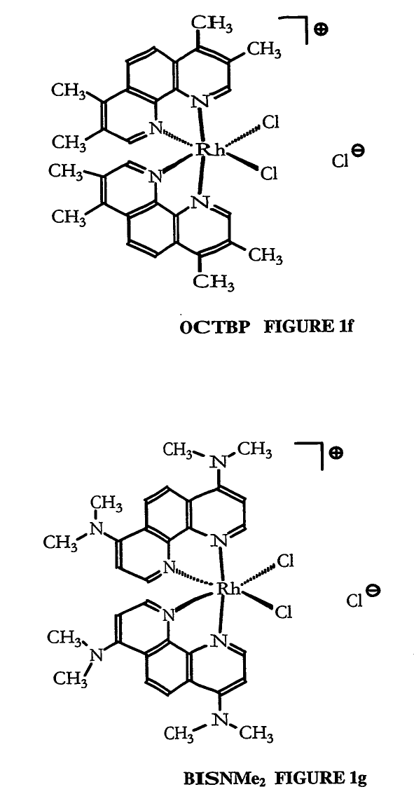

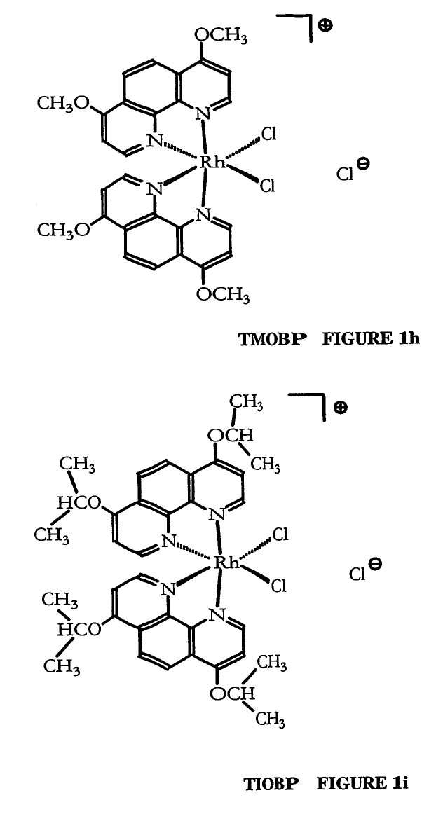

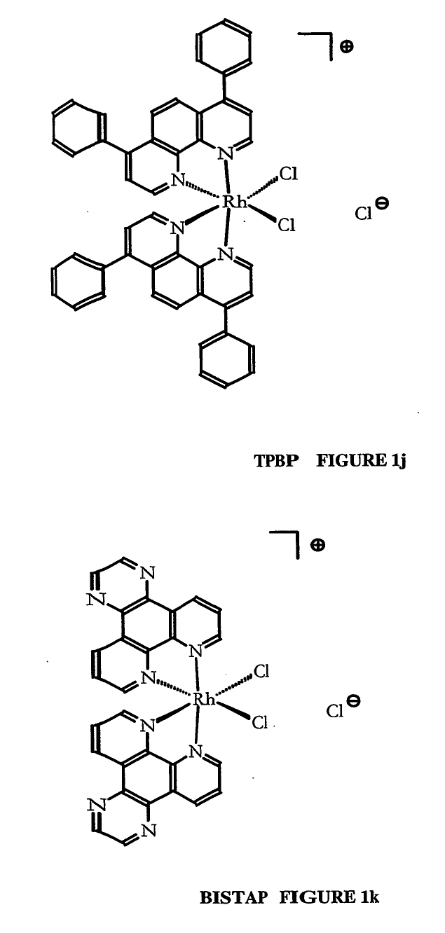

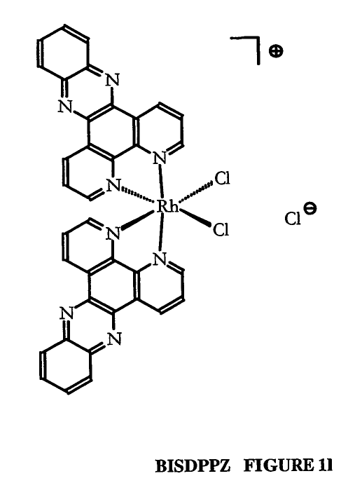

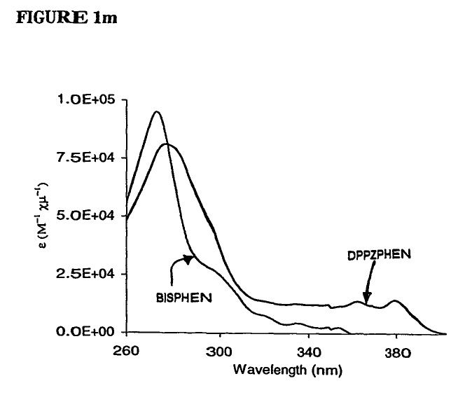

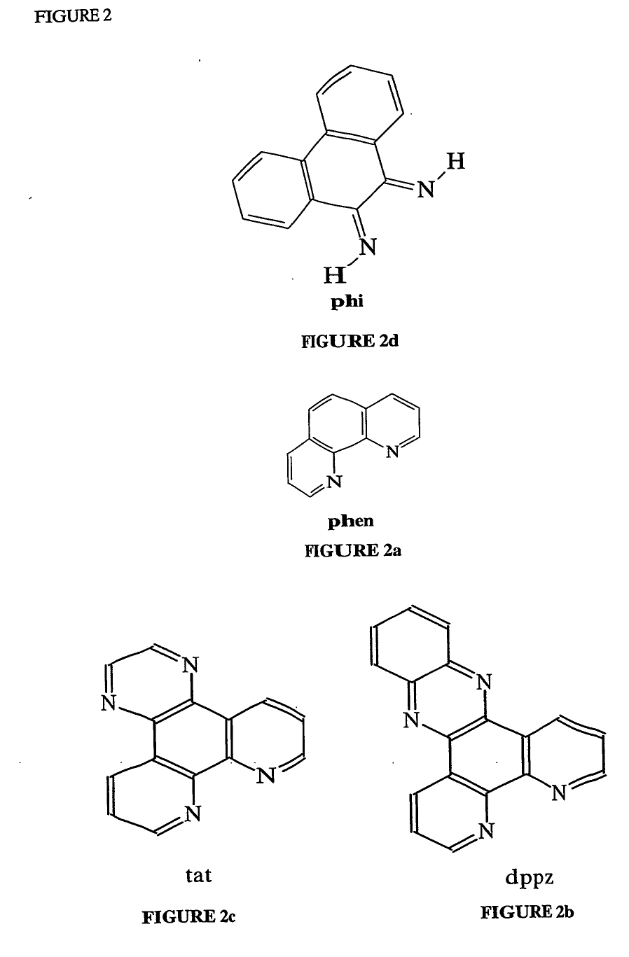

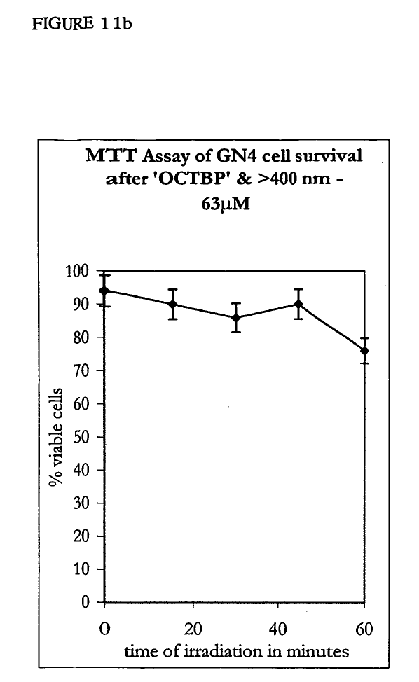

The present invention provides transition metal compounds that have great potential as photoactivated anti-tumor and anti-viral agents. Bisbipyridyl rhodium (III) compounds of the invention (also referred to herein as “rhodium (III) compounds”) include compounds having formulae I, II, III and IV, as follows: 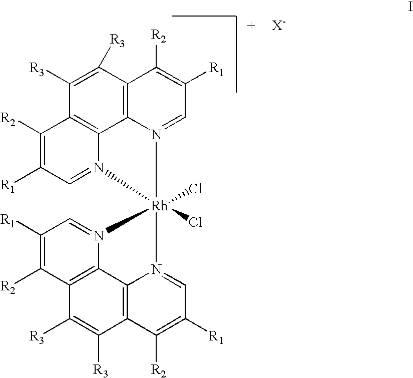
wherein R₁, R₂ and R₃ are each independently selected from the group consisting of an alkyl group, an alkenyl group, an alkynyl group, a nitrile, an azide, an aryl group, an aralkyl group, a heteroaryl group, a hydroxy group, an alkoxy group, an aryloxy group, an amine group, and a hydrogen atom, or any two of R₁, R₂ and R₃ together form an aryl or heteroaryl ring; and wherein X is a counterion; 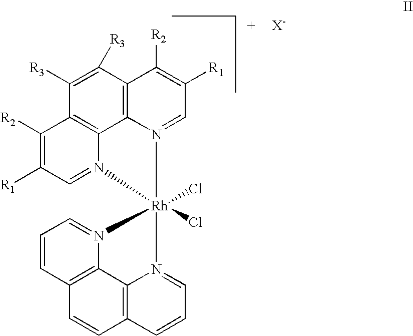
wherein R₁, R₂ and R₃ are each independently selected from the group consisting of an alkyl group, an alkenyl group, an alkynyl group, a nitrile, an azide, an aryl group, an aralkyl group, a heteroaryl group, a hydroxy group, an alkoxy group, an aryloxy group, an amine group, and a hydrogen atom, or any two of R₁, R₂ and R₃ together form an aryl or heteroaryl ring, and wherein X is a counterion. Optionally, where R₁=R₃=H, R₂ is neither methyl nor phenyl; 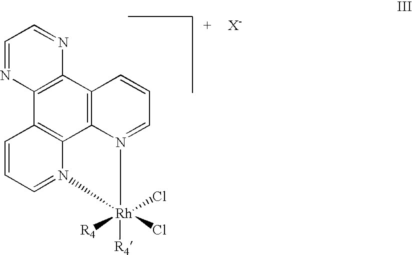
where R₄ and R₄′ together form a phen ligand, yielding cis-dichloro{2,3-di(2-pyridyl)quinoxaline}{1,10-phenanthroline}rhodium (III)chloride (TAPPHEN) or a “tap” ligand, yielding cis-dichlorobis{2,3-di(2-pyridyl)quinoxaline} rhodium (III) chloride (BISTAP, FIG. 1k); or 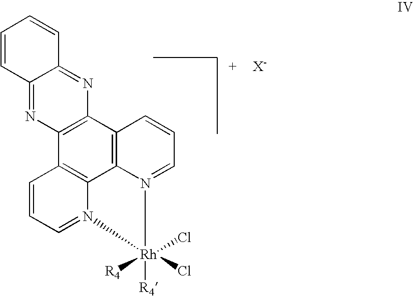
wherein R₄ and R₄′ together form a phen ligand, yielding cis-dichloro(dipyrido[3,2a-2′3′c]phenazine)(1,10-phenantroline) rhodium(III) chloride (DPPZPHEN) (FIG. 1c) or a dppz ligand (FIG. 2b), yielding cis-dichlorobis{dipyrido(3,2-a: 2′,3′-c)phenazine}rhodium (III) chloride (BISDPPZ) (FIG. 11).

Methods for making the rhodium (III) compounds of formula I, II, III and IV are also encompassed by the invention and are illustrated in the Examples that follow.

The bisbipyridyl ligands in formulas I or II may contain any organic substituent group which does not eliminate the phototoxicity of the compound. One skilled in the art can readily determine the suitability of a particular substituent group or groups empirically using any of the standard assays for determining the level of active intracellular pathogenic contaminants. Illustrative examples of organic substituents include, but are not limited to, alkyl groups, alkenyl groups, alkynyl groups, hydroxy groups, alkoxy groups, aryl groups, heteroaryl groups, aryloxy groups, heteroaryloxy groups, nitro groups, amine groups, amide groups, alkylcarboxyl groups, arylcarboxyl groups, aralkyl groups, cyano groups, azide groups, haloalkyl groups, and haloaryl groups. Preferable organic substituents include alkyl groups, such as methyl, ethyl, and propyl groups, alkenyl groups, such as ethenyl groups, alkynyl groups, such as acetenyl groups, and amine groups, such as monomethylamine and dimethylamine groups.

As used herein, the term “alkyl” or “alkyl group” means a straight or branched chain hydrocarbon substituent having from 1 to 10 carbon atoms, preferably 1 to 4 carbon atoms, and more preferably 1 or 2 carbon atoms, such as methyl, ethyl, and the like. Likewise the term “alkenyl” or “alkenyl group” means a straight or branched chain hydrocarbon substituent having from 2-10 carbon atoms and at least one double bond, such as ethenyl or propenyl and the like. The term “alkynyl” or “alkynyl group” as used herein means a straight or branched chain hydrocarbon substituent having from 2-10 carbon atoms and at least one triple bond, such as ethynyl or propynyl and the like. The term “aryl” or “aryl group” means a monocyclic or bicyclic aromatic hydrocarbon substituent having from 6-12 carbon atoms in the ring(s), such as phenyl or naphthyl and the like. The term “aralkyl” or “aralkyl group” means a straight or branched chain hydrocarbon substituent having from 1 to 6 carbon atoms bound to a monocyclic or bicyclic aromatic hydrocarbon substituent having from 6-12 carbon atoms in the ring(s), such as benzyl or 2-phenylethyl and the like; and the term “heteroaryl” or “heteroaryl group” means a monocyclic, bicyclic, or tricyclic aromatic substituent having from 4-11 carbon atoms and at least one hetero atom (i.e. an oxygen atom, a nitrogen atom and/or a sulfur atom) in the ring(s), such as thienyl, furyl, pyranyl, pyridyl, quinolyl, and the like.

Alkyl groups, because they are generally electron-donating when in proximity to an electron deficient species, such as a transition metal like rhodium, are preferred aromatic ring substituents for the phototoxic compounds of the invention. Without intending to be bound by theory, alkyl substituents may facilitate intercalation and/or groove binding of the rhodium (III) compounds of the invention into nucleic acids and may also facilitate photolytic cleavage and hydrolysis of the Rh-Cl bond, which is necessary in order for the compound to form a covalent adduct with the nucleic acid. The increase in hydrophobicity due to the alkyl substituents may also aid in membrane transport and association with the nucleic acid.

Counterion X can be a monovalent anion, such as a halide, preferably chloride or bromide, or a polyvalent anion, such as sulfate, phosphate, or an anionic organic moiety, such as carbonate, acetate, citrate or tartrate. Preferably, the counterion is chloride.

The rhodium (III) compounds of the invention are stable in aqueous solution in the dark but are preferably stored in the dark as a solid. Preferred compounds include (a) a compound having formula I or II wherein R₁=R₂=H and R₃=(C1-C4) alkyl; preferably formula I wherein R₁=R₂=H and R₃=CH₃ (cis-dichlorobis(5,6-dimethyl-1,10-phenanthroline) rhodium(III) chloride; 56TMBP, FIG. 1e); (b) a compound having formula I or II wherein R₁=R₂=(C1-C4) alkyl and R₃=H; preferably formula I wherein R₁=R₂=CH₃ and R₃=H (cis-dichlorobis(3,4,7,8-tetramethyl-1,10-phenanthroline) rhodium(III) chloride; OCTBP, FIG. 1f); (c) a compound having formula I or II wherein R₁=R₃=H and R₂=N—(C1-C4)alkyl; preferably formula I wherein R₁=R₃=H and R₂=N(CH₃)₂ (cis-dichlorobis{3,7(N,N-dimethylamino)-1,10-phenanthroline) rhodium(III) chloride; BISNMe2, FIG. 1g); (d) compounds having formula I or II wherein R₁=R₃=H and R₂=O—(C1-C4) alkyl; preferably formula I wherein R₁=R₃=H and R₂=O—CH₃ (cis-dichlorobis(3,7-dimethoxy-1,10-phenanthroline) rhodium(III) chloride; TMOBP, FIG. 1h) or O—(CH₂)(CH₃)₂ (cis-dichlorobis(3,7-diisopropoxy-1,10-phenanthroline) rhodium(III) chloride; TIOBP, FIG. 1i); and (e) compounds having formula I or II wherein R₁=R₃=H or (C1-C4)phenyl, preferably formula I wherein R₁=R₃=H and R₂=phenyl (cis-dichlorobis(4,7-diphenyl-1,10-phenanthroline) rhodium(III) chloride; TPBP, FIG. 1j). Preferably, for a compound having formula I, where R₁=R₃=H, R₂ is neither methyl nor phenyl.

The compounds of the invention possess one or more of the following desirable properties: (a) they are capable of photonicking naked DNA and/or RNA; (b) they can permeate bacterial and/or eukaryotic cell membranes; (c) they associate with DNA and/or RNA in the dark; and (d) they are phototoxic to bacterial and/or eukaryotic cells. Preferably the compounds of the invention elute at about 15 minutes from a C8 reversed phase high performance liquid chromatography column in 45% acetonitrile in aqueous buffer, such as 100 mM ammonium acetate at pH 5.4. They are capable of reducing the level of one or more active extracellular or intracellular, pathogens, such as viral or bacterial contaminants, in a biological composition. Bioactive rhodium (III) compounds of the invention that are not able to permeate cell membranes may be delivered to a cell using a vehicle such as a liposome, an organic polymer, or a membrane receptor-targeting ligand or membrane transport molecule such as a peptide. Carrier-mediated delivery allows increased accumulation of the rhodium (III) compound at the targeted site. The delivery vehicle can be bound covalently or noncovalently to the bioactive compound. Photoimmunotargeting uses monoclonal antibodies that recognize tumor antigens. Ligands against receptors that are upregulated in tumor cells can also be utilized as delivery vehicles. Examples of targets include the low-density lipoprotein receptor, the peripheral benzodiazepine receptor, and the estrogen receptor.

Illustrative examples of pathogenic organisms that can be inactivated by phototreatment with the compound of the invention include, but are not limited to bacteria, such as Streptococcus, Staphylococcus, Escherichia, and Bacillus; viruses, such as human immunodeficiency viruses (HIV-1; HIV-2) and other retroviruses, human T-cell lymphotorophic virus-I (HTLV-I), herpes viruses, paramyxoviruses, cytomegaloviruses, hepatitis viruses (including hepatitis A, B and C), Vesicular stomatitis virus (VSV), Moloney sarcoma virus, Sindbis virus, Epstein Barr viruses, lactate dehydrogenase elevating virus, rhabdoviruses, leukoviruses, myxoviruses, alphaviruses, arboviruses (group B), paramyxoviruses, arenaviruses and coronaviruses, parvovirus, polio virus, bacteriophage M13 and satellite adeno-associated virus (AAV), pox viruses, and toga viruses; and parasites, such as malarial parasites, including Plasmodium species, and trypanosomal parasites.

Preferably, the compounds of the invention minimally bind proteins present in the biological material, so as maintain maximum bioavailablity for their intended purpose of binding and inactivating pathogenic nucleic acids.

The method for inactivating pathogenic contaminants, as provided by the invention, involves contacting the biological material with a bisbipyridyl rhodium (III) compound described herein, followed by irradiation of the treated sample to inactivate the pathogenic contaminant. Pathogenic contaminants include not only pathogenic organisms as exemplified above but also dysplastic or cancerous cells, leukocytes, and any other potentially pathogenic or otherwise undesired nucleic acid containing material.

The method can be practiced using any biological material, but is especially well suited for use in decontaminating physiological samples or materials such as blood, semen, ascites fluid, milk, lymphatic fluid, an organ, a tissue, or a hybridoma cell line. Blood and blood products or fractions include whole blood as well as such as cellular blood components, including red blood cell concentrates, leukocyte concentrates, and platelet concentrates and extracts; liquid blood components such as plasma and serum; and blood proteins such as clotting factors, enzymes, albumin, plasminogen, and immunoglobulins, or mixtures of cellular, protein and/or liquid blood components. Details regarding the make-up of blood, the usefulness of blood transfusions, cell-types found in blood and proteins found in blood are set forth in U.S. Pat. No. 5,232,844. Techniques regarding blood plasma fractionation are generally well known to those of ordinary skill in the art and an excellent survey of blood fractionation also appears in Kirk-Othmer's Encyclopedia of Chemical Technology, Third Edition, Interscience Publishers, Volume 4.

An important difference between various blood fractions is cellular in nature. Plasma is an aqueous suspension of proteins, and microbial pathogens are thus also in suspension. Platelets, erythrocytes (red blood cells) and leukocytes (white blood cells) on the other hand are cells, and microbial pathogens may be intracellular, extracellular or both. Because the rhodium (III) complexes of the invention either pass through cell membranes or can be complexed with a delivery vehicle that assists them in passing through cell membranes, they are effective against both intracellular and extracellular pathogens.

It should also be noted that leukocytes contain nucleic acid whereas the other cell types, and plasma, do not. Since the method of the invention is phototoxic to leukocytes, it can be practiced on blood samples that include leukocytes as long as damage to these cells is either desired or immaterial. Leukocytes, for example, may be associated with infectious agents, therefore white cell reduction (leukodepletion) is an important process and can be achieved using the method of the invention.

Furthermore, some blood products, such as red blood cell concentrates, include hemoglobin, which may interfere with the efficacy of the method of the invention due to significant absorption at wavelengths typically used to photoactivate the rhodium (III) compound of the invention. This problem can be overcome by irradiating the sample using a longer wavelength light, or by including a sensitizer molecule in the photoreaction, as discussed in more detail below.

Decontamination of blood and blood products according to the invention represent a significant advance in maintaining and improving public health by insuring the safety of the blood supply. However this method can also be used to decontaminate a patient's own blood. Blood is removed from the patient, decontaminated using the method of the invention, then returned to the patient in a process known as “photophoresis.” This method can, for example, be used to treat lymphoma. The blood may be further processed prior to return to the patient, for example by concentrating it or removing the phototoxic agent.

As noted above, the method of the invention is not limited to decontamination of blood or blood products. The method can be employed, for example, to decontaminate compositions containing non-blood components such as normal or cancerous cells, or to treat a patient suffering from disease, especially localized disease, in vivo or ex vivo. Diseases that can be treated include bacterial, viral and protozoan diseases as well as somatic disease such as neoplasia, dysplasia and cancer.

The rhodium (III) compound can be delivered to the patient in any convenient manner. For example, it can be injected into the bloodstream and absorbed by cells all over the body, or preferentially by targeted disease cells. It can likewise be delivered by oral administration, topical application, perfusion and the like. In one embodiment of the method of treating a patient according to the invention, the photoactive rhodium (III) compounds are complexed with another molecule, such as an antibody or a receptor ligand, that specifically targets a tumor cell. When the treated disease cells, such as cancer cells, are exposed to light of the appropriate wavelength, the phototoxic agent is activated and destroys the treated diseased cells. Light exposure must be either timed carefully so that it occurs when most of the photosensitizing agent has left healthy cells but is still present in the diseased cells, or must be specifically directed to a disease site, such as a tumor.

Light used to photoactivate the rhodium (III) compound, particularly laser light, can be directed through a fiber-optic (a very thin glass strand). The fiber-optic is placed close to the cancer to deliver the proper amount of light. For example, the fiber-optic can be directed through a bronchoscope into the lungs for the treatment of lung cancer, or through an endoscope into the esophagus for the treatment of esophageal cancer. Because laser light typically cannot pass through more than about 3 centimeters of tissue, the method is preferably used to treat tumors on or just under the skin, or on the lining of internal organs.

Importantly, the bioactivity of the compounds of the invention was found to be unaffected by the presence or absence of oxygen. This suggests the exciting possibility that such compounds and their analogs might be effective against hypoxic tumor cells. Furthermore, the fact that the method of the present invention apparently does not create reactive oxygen species (ROS) implies that the high levels of virus inactivation will be achievable with less damage to platelet function than occurs using oxygen-dependent photochemistry for pathogen inactivation. See also U.S. Pat. No. 6,087,141, Margolis-Nunno et al. Indiscriminate photosensitized damage of blood components represents one of the major drawbacks of currently employed photodecontaminating drugs (Santus et al., Clin. Hemorheology and Microcirculation, 18:299-308 (1998)) that rely on the formation of singlet oxygen for their effectiveness (Wainwright, Current Med. Chem., 9:127-143 (2002)). For example, the formation of singlet oxygen is known to damage cell membranes. In the present invention, it is expected that less generalized damage will occur in both oxygenated and hypoxic environments, due to the fact that little or no singlet oxygen is produced.

To decontaminate or otherwise treat a biological sample, the biological material is contacted with an effective amount of the rhodium (III) compound of the invention, then irradiated for a time with light having a preselected wavelength or range of wavelengths in a manner sufficient to reduce the level of active pathogenic contaminants therein. Optionally, the rhodium (III) compound is removed from the biological material, for example via affinity or ion exchange chromatography, dialysis or microfiltration.

Irradiation exposure periods of between 30 and 45 minutes are generally sufficient, but depend on the conditions used. The wavelength used to irradiate the rhodium (III) compound is typically 300 nm to 400 nm, since the rhodium (III) compounds of the invention typically have absorption maxima between 310 nm and 400 nm. It is preferable to use UVA wavelength light (320 nm-400 nm) to minimize cell damage; however, reliable narrow band UVB monochromatic lamps are available (311/312 nm) and can be effectively used in the method of the invention as well. The extinction coefficients for the rhodium (III) compounds of the invention at 311 nm vary from compound to compound but are generally in the neighborhood of 10,000 to 20,000 M⁻¹cm⁻¹. The light source can be a broad band or narrow band light source. Examples of light sources include tungsten, mercury, or xenon arc lamps, as well as lasers. Light can be filtered using a bandpass filter. Rhodium (III) compounds of the invention that contain amines typically have a higher extinction coefficient than the other rhodium (III) compounds and therefore can be irradiated for a shorter length of time.

It has also been found that irradiation of the rhodium (III) compound with longer wavelength light (i.e., a >400 nm bandpass filter) still resulted in phototoxicity. Thus, the method includes irradiation of samples at a longer wavelength, which potentially results in less damage to platelets and other sensitive blood components.

It should be noted that red blood cell decontamination methods using photochemicals have previously encountered a problem due to the absorbency of light by hemoglobin at wavelengths necessary to activate such compounds (e.g., about 400 nm-550 nm). Thus, blood decontamination method of the present invention is preferably applied to blood products that do not contain red blood cells, such as platelet concentrates, plasma or cell-free blood proteins

However, co-treatment of a hemoglobin-containing blood product with an photosensitizer molecule may allow the rhodium (III) compounds of the invention to be successfully used to decontaminate blood products containing hemoglobin provided the sensitizer molecule absorbs light of a significantly higher wavelength. The wavelength used to excite the sensitizer molecule is preferably above about 550 nm and is a wavelength at which the sensitizer compound exhibits an extinction coefficient of at least 250 M⁻¹cm⁻¹, more preferably at least 1000 M⁻¹cm⁻¹, even more preferably at least 10000 M⁻¹cm⁻¹. Preferably the sensitizer molecule is water soluble. Convenient sensitizer molecules that have sufficient absorptivity at these longer wavelengths include many well-known dyes that intercalate nucleic acids.

The sensitizer molecule can activate the rhodium (III) compound via either electron transfer, which involves an actual oxidation/reduction reaction between the sensitizer and the rhodium (III) complex and/or the biological material, or energy transfer, in which the sensitizer molecule indirectly activates the rhodium (III) compound through triplet excited state energy transfer. A sensitizer molecule that works by way of energy transfer preferably has a triplet energy in excess of 45 kcal/mol. A sensitizer molecule that works by way of electron transfer, such as an anionic dye, is preferably readily oxidizable, thereby being capable of reducing the metal complex.

An example of a sensitizer molecule that works by way of electron transfer is methylene blue (and derivatives thereof), which can be adequately excited by light having a wavelength of about 550 nm to 730 nm. When irradiated using a wavelength of 578 nm or 633 nm in the presence of BISPHEN, for example, methylene blue led to irreversible photobinding of both reagents to DNA (Mohammad et al., Photochem. Photobiol., 71(4):369-381 (2000)). Methylene blue is known to be phototoxic in its own right, albeit using a different mechanism than that attributable to the rhodium (III) compound, so in addition to serving as a sensitizer molecule it participates directly in the phototoxic reaction. Acridine orange, which has a triplet energy of 206 kJ/mol (about 50 kcal/mol), likely functions by way of energy transfer when used as a sensitizer molecule. Although it has its maximum absorption at 430 nm, the extinction coefficient at that wavelength is 27,000 M⁻¹cm⁻¹ and the molecule still exhibits appreciable absorption at wavelengths of 550 nm and higher.

### EXAMPLES

The present invention is illustrated by the following examples. It is to be understood that the particular examples, materials, amounts, and procedures are to be interpreted broadly in accordance with the scope and spirit of the invention as set forth herein.

### Example I

### Synthesis of cis-dichloro(dipyrido[3,2a-2′3′c]phenazine)(1,10-phenantroline) rhodium(III) chloride (i.e., cis-Rh(dppz)(phen)Cl₂⁺ or DPPZPHEN)

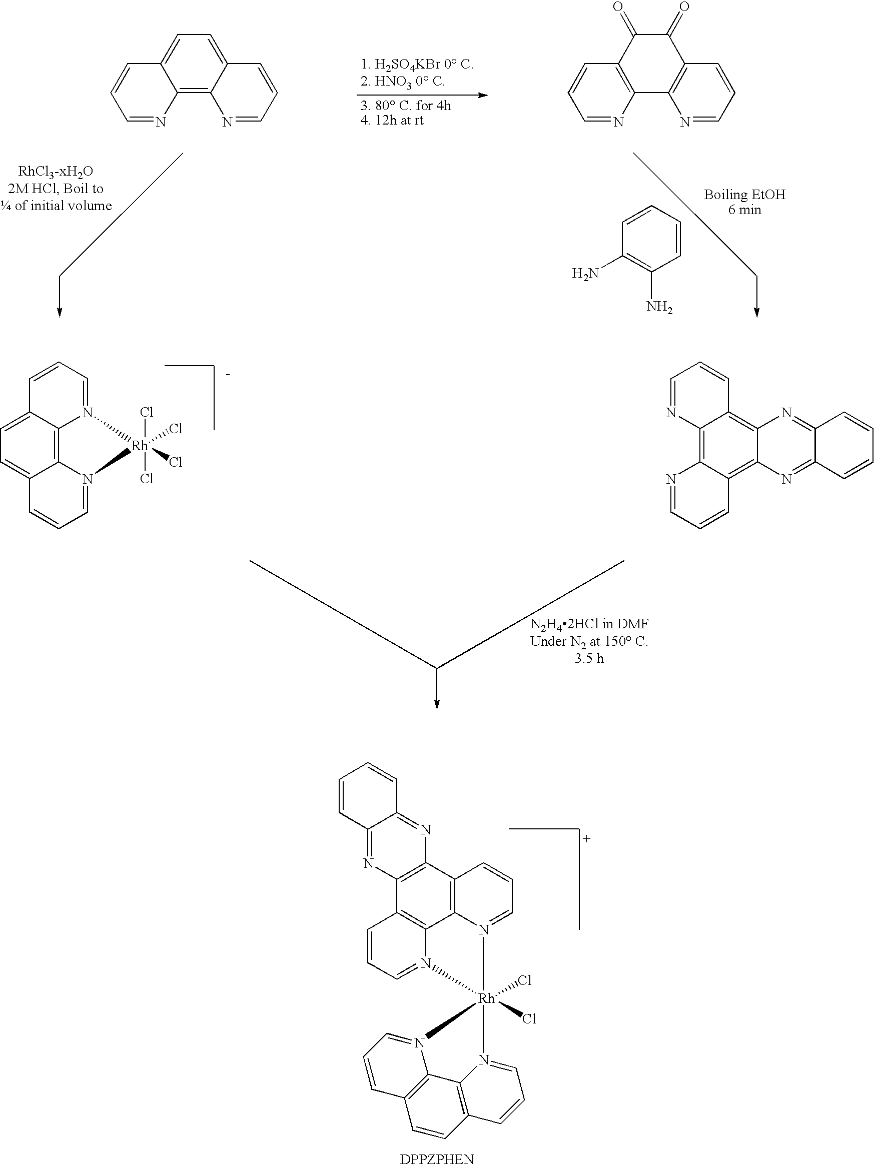

1,10-phenanthroline-5,6-dione and K[Rh(phen)Cl₄] (monophen) were prepared from 1,10-phenanthroline. 1,10-phenanthroline-5,6-dione was synthesized according to Bull. Chem. Soc., 65:1006-1011 (1992). A round-bottom flask containing 0.5202 g (2.89 mmol) of phen hydrate and 3.0148 g of potassium bromide was placed in an ice bath. Concentrated sulfuric acid (10 ml) was added in small portions and then 5 ml of concentrated nitric acid was added. The resulting solution was heated for 2 hours at 105-115° C., then cooled to room temperature and poured into 200 ml of water. The solution was neutralized with sodium hydroxide (10 mM) and then was extracted with dichloromethane. The organic layer was dried, filtered and evaporated to give 0.538 g of yellow solid (yield 88.6%).

The dione was condensed with 1,2-phenylenediamine to form dipyrido[3,2-a:2′,3′-c]phenazine (dppz). A mixture of 108.2 mg (0.515 mmol) of 1,10-phenanthroline-5,6-dione in 5 ml ethanol and 106.5 mg (0.986 mmol) in 5 ml of ethanol was refluxed for 40 minutes. The solution was cooled to room temperature and left in the refrigerator, after which the yellow solid precipitated. The solid (101 mg) was recrystallized by ethanol-water (yield: 43%).

The ligand dppz (Dikeson et al., Aust. J. Chem., 23:1023-1027 (1970)) (30 mg; 0.106 mmol) and N₂H₄.HCl (2 mg; 0.03 mmol) were, placed in a 50 mL three neck round bottom flask fitted with a condenser. DMF (5 mL) was added and the resultant slurry was degassed with nitrogen for 20 minutes. K[Rh(phen)Cl₄].H₂O (51.3 mg; 0.106 mmol) in DMF (10 mL) was degassed for 20 minutes with N₂ under constant stirring to form an orange suspension that was transferred to the dppz slurry using a stream of N₂. The temperature was slowly increased to between 110° C. and 150° C., while gently bubbling with N₂, until all the solids were dissolved and a bright orange solution was formed. The temperature was further increased and the solution allowed to reflux for 3.5 hours, after which it had turned a gasoline-yellow color. The solution was cooled to RT with stirring and transferred to a 250 mL Erlenmeyer flask. Ether (about 60 mL) was added and the resultant beige precipitate was collected by vacuum filtration. This was washed immediately with copious amounts of ethanol, dissolved in 50 mL of boiling water and filtered. A saturated solution of KCl was added until a precipitate start to form, and the mixture then left overnight in a refrigerator. A beige precipitate of DPPZPHEN (55.8 mg; yield: 76.5%) was collected by vacuum filtration that was >98% pure by HPLC. UV-Vis (50 mM pH 7.0 phosphate buffer); λ nm (ε M⁻¹cm⁻¹): 277 (77,798); 362 (13,864); 380 (14,400). FAB MS (z/e): 634.7, (Rh(dppz)(phen)Cl₂⁺) 599.7, (Rh(dppz)(phen)Cl⁺) 564.7, (Rh(dppz)(phen)⁺). HRMS (m/z): found 635.0019; calc. 635.0025. ¹HNMR (DMSO-d₆, 500 MHz) δ: 10.13 (d, J=5.25, 1H); 10.05-10.02 (dd, 2H); 9.65 (d, J=8.16, 1H); 9.31 (d, J=8.15, 1H); 8.92 (d, J=8.09, 1H); 8.69-8.66 (dd, 1H); 8.57-8.53 (m, 3H); 8.47-8.42 (m, 2H); 8.22-8.15 (m, 4H); 7.93-7.90 (dd, 1H); 7.82-7.79 (dd, 1H).

### Example II

### Synthesis of cis-dichlorobis{dipyrido(3,2-a: 2′,3′-c)phenazine)rhodium (III) chloride (BISDPPZ)

132.5 mg of RhCl₃.3H₂O (about 0.5 mmol) (Pressure Chemical Company, Pittsburgh, Pa.) was dissolved in 50 mL of deionized water by boiling and continuously stirring. 282 mg of dppz was dissolved in 50 mL of absolute ethanol by boiling. The hot ethanolic solution of dppz was added dropwise to the hot aqueous solution of RhCl₃ while continuously stirring. The container of the ligand was washed with further 50 mL of ethanol and it was added to the RhCl₃ solution. The resulting mixture was further boiled for 15 minutes vigorously until the volume of the solution reaches up to 20 mL. The resulting solution was cooled initially to room temperature and then to 5° C. for 12 hours to yield beige color solid. The solid was recrystallized in ethanol to give tiny yellow crystals. ¹H-nmr(dmso-d₆): δ 10.18 (d, J=5.5 Hz, 2H), 10.08 (d, J=8.24 Hz, 2H), 9.70 (d, J=8.24 Hz, 2H), 8.73 (dd, J=8.24 Hz, J=5-5 Hz, 2H), 8.60(dd, J=6.41 Hz, J=2.13 Hz, 2H), 8.51(dd, J=7.32 Hz, J=32, 2H), 8.24 (m, 6H), 7.96(dd, J=8.24 Hz, J=5.64 Hz, 2H). LRMS (ESI) for positive ion 737/739. HRMS (ESI) for positive ion 737.0243 (Calculated), 737.0248 (Actual).

### Example III

### Synthesis of cis-dichlorobis(2,3-di(2-pyridyl)quinoxaline} rhodium (III) chloride (BISTAP)

53 mg of RhCl₃.3H₂O (about 0.215 mmol) was dissolved in 15 mL of water by boiling. 100 mg of tetraazatriphenylene (TAP) was dissolved in 10 mL of absolute ethanol by boiling. The hot boiling ethanolic solution was added to the hot aqueous solution of RhCl₃ dropwise. The resulting mixture was boiled for 10 minutes. After cooling the mixture, catalytic amount of NH₂NH₂.HCl was added to the mixture and it was boiled for another 15 minutes. Cooling the mixture to room temperature, followed by further cooling to 5° C. gave yellow crystals of BISTAP (145 mg). The solid was filtered under vacuum, washed with 3×50 mL of ice-cold water followed by 3×10 mL of ice-cold ethanol. The crystals were air dried under vacuum to give 140 mg of yellow powder (yield: 89%). ¹H-nmr(dmso-d₆): δ 10.2(d, J=6.18 Hz, 2H), 10.0(dd, J=8.30 Hz, 0.46 Hz, 2H), 9.65(dd, J=8.30 Hz, 1.14 Hz, 2H), 9.45 (dd, J=10.70 Hz, 2.16 Hz, 4H), 8.70(dd, J=8.34 Hz, 5.5 Hz, 2H), 8.25(d, J=5.45 Hz, 2H), 7.95(dd, J=8.30 Hz, 5.49 Hz, 2H). ¹³C-nmr(dmso-d₆): δ 154.224, 153.628, 148.783, 148.008, 147.488, 147.404, 139.371, 139.166, 137.090, 136.382, 130.480, 130.053, 128.994, 128.282. LRMS (ESI) for positive ion 637/639. HRMS(ESI) for positive ion 636.9930 (Calculated), 636.9940 (Actual).

### Example IV

### Synthesis of cis-dichlorobis(3,4,7,8-tetramethyl-1,10-phenanthroline) rhodium(III) chloride (OCTBP)

123 mg (0.5 mmol) RhCl₃.3H₂O was dissolved in 15 mL of water by boiling. 240 mg (1 mmol) of 3,4,7,8-tetramethyl-1,10-phenanthroline (Sigma Aldrich) was dissolved in 15 mL absolute ethanol by boiling. The ethanol solution of the ligand was added to the hot aqueous solution of RhCl₃ dropwise. The resulting mixture was boiled for 15 minutes. After cooling the mixture to room temperature, 20 mg of NH₂NH₂.HCl was added to the mixture and boiled for another 25 minutes. Cooling the mixture to room temperature followed by further cooling to 5° C. resulted yellow crystals of OCTBP. The solid was filtered, washed with 3×50 mL of ice-cold water followed by 3×10 mL of ice-cold ethanol. The crystals were air dried, then dried under vacuum to give 300 mg of OCTBP (yield: 81%). ¹H-nmr(200 MHz, DMSO-d₆): δ 9.7(s, 2H), 8.65(d, J=9.4 Hz, 2H), 8.5(d, J=9.48 Hz, 2H), 7.75(s, 2H), 3.05(s, 6H), 2.8(s, 6H), 2.7(s, 6H), and 2.35(s, 6H). ¹³C-nmr(200 MHz, DMSO-d₆): δ 152.51, 151.21, 148.50, 148.32, 145.73, 145.02, 135.94, 135.87, 129.93, 129.52, 125.02, 124.56, 18.45, 17.62, 15.40, and 14.99. LRMS/ESI: positive ion m/z 645/647. UV/Visible (in water): λₘₐₓ=312 nm (18169 M⁻¹cm⁻¹), 336 nm (5025 M⁻¹cm⁻¹), 353 nm (1560 M⁻¹cm⁻¹).

### Example V

### Synthesis of cis-dichlorobis(5,6-dimethyl-1,10-phenanthroline) rhodium(III) chloride (56TMBP)

0.2 g of RhCl₃.3H₂O (about 0.85 mmols) was dissolved in 15 mL of water by boiling. 5,6-dimethyl-1,10-phenanthroline (0.35 g; 1.7 mmols) was dissolved in 15 mL of absolute ethanol. Boiling ethanolic solution of the ligand was added to the hot aqueous solution of RhCl3 carefully. The resulting mixture was boiled vigorously until the volume of the solution became up to about 10 mL (about 10 minutes). The heat source was removed and it was allowed to cool down to room temperature. Then it was kept it in the refrigerator at 5° C. for 12 hours. The bright yellow crystals were collected by vacuum filtration, washed with ice cold water (3×10 mL), then by ice-cold ethanol (3×10 mL), followed by diethylether (3×10 mL), air dried and dried under vacuum to yield 0.44 g of 56TMBP (85%). ¹H-nmr(200 MHz, DMSO-d₆): 10.0(d, J=5.27 Hz, 2H), 9.38(d, J=8.2 Hz, 2H), 8.97(d, J=8.65 Hz, 2H), 8.52(dd, J=8.52 Hz, 5.39 Hz, 2H), 7.95(d, J=5.27 Hz, 2H), 7.73(dd, J=8.53 Hz, 5.4 Hz, 2H), 2.95(s, 6H), and 2.80(s, 6H). ¹³C-nmr(200 MHz, DMSO-d₆): 151.81, 150.66, 145.87, 145.24, 137.77, 137.15, 133.15, 132.87, 131.71, 131.32, 127.47, 126.88, 15.53, 15.37. LRMS(ESI) for positive ion: 589/591. HRMS(ESI) for positive ion: 589.0433(calculated) and 589.0436(actual).

### Example VI

### Syntheses of 4,7-disubstituted-1,10-phenanathrolines and tetrasubstituted “BISPHEN” analogs (BISNMe2, TMOBP and TIOBP)

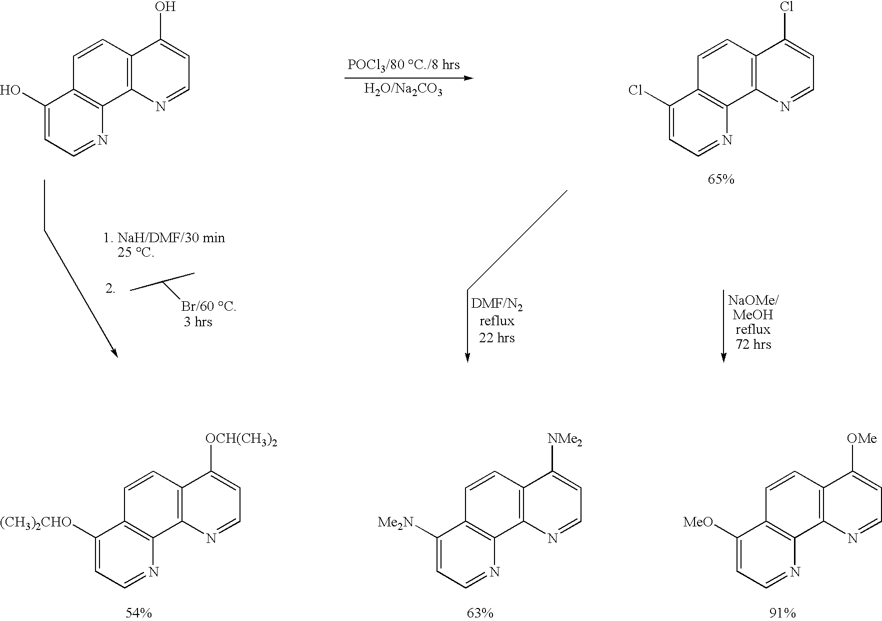

### Preparation of 4,7-dichloro-1,10-phenanthroline

A mixture of 2.80 g (13 mmol) of 4,7-dihydroxy-1,10-phenanthroline and 40 mL of phosphorous oxychloride were stirred under N₂ at 80° C. for 7 hours. The cooled reaction mixture was added into 200 mL of well-stirred ice-cold water dropwise. The solution was carefully neutralized by adding solid Na₂CO₃ in small portions with vigorous stirring. The resulting mixture was extracted with chloroform (3×150 mL). The combined extracts were washed with water, dried with Na₂SO₄ and evaporated in vacuo. 2.1 g (yield: 65%) of beige color solid was obtained.

1H-nmr(200 MHz, CDCl₃): δ 9.0(d, 4.84 Hz, 2H), 8.3(s, 2H), 7.7(d, 4.8 Hz, 2H) ¹³C-nmr(200 MHz, CDCl₃): δ 150.53, 147.0, 143.13, 127.10, 124.10, 123.48 MS/EI: M⁺=248/250/252 (base peak)-dichloro pattern; M⁺-Cl=213/215 MS/CI: M⁺+H=249/251/253(base peak).

### Preparation of 4,7-bis(dimethylamino)-1,10-phenanthroline

A suspension of 590 μg (2.4 mmol) of 4,7-dichloro-1,10-phenanthroline in 20 mL of DMF was refluxed under N₂ for 30 hours. After the solvent was evaporated under vacuum, the orange powder was treated with a mixture of 20 mL of 1M NaOH and 25 mL of THF. The combined THF fractions were dried over Na₂SO₄, and the solvent was evaporated. The crude product was dissolved in 20 mL of dichloromethane and washed with 2×10 mL of 1M NaOH. The dichloromethane was evaporated to give brown residues of 400 mg (yield: 63%). ¹H-nmr(200 MHz, DMSO): δ 8.75(d, 5.25 Hz, 2H), 8(s, 2H), 7.15(d, 5.17 Hz, 2H), 3.10(s, 6H). ¹³C-nmr(200 MHz, DMSO): δ 157.48, 149.89, 147.57, 121.51, 120.90, 109.90, 43.94. MS/EI: M⁺=266(base peak). MS/CI: M⁺+H=267(base peak).

### Preparation of 4,7-dimethoxy-1,10-phenanthroline

300 mg (1.2 mmol) of 4,7-dichloro-1,10-phenanthroline and 600 mg (11 mmol) of NaOMe were dissolved in 40 mL of HPLC grade methanol and refluxed for 60 hours, after which it was concentrated in vacuum up to 15 mL and 20 mL of ice-cold water was added to it. The suspension was stored overnight at 4° C. for complete precipitation of the product. The precipitate was filtered off, washed with 3×15 mL of water and dried under vacuum to yield 313 mg of yellow powder (yield: 91%). ¹H-nmr(200 MHz, CDCl₃): δ 9.0(d, 5.33 Hz, 2H), 8.2(s, 2H), 7.0(d, 5.25 Hz, 2H), 4.10(s, 6H). ¹³C-nmr(200 MHz, CDCl₃): δ 162.73, 151.69, 147.35, 121.42, 119.45, 103.24, 56.34. MS/EI: M⁺=240(base peak), M⁺-CH₃=225. MS/CI: M⁺+H=241(base peak).

### Preparation of 4,7-diisopropoxy-1,10-phenanthroline

4,7-dihydroxy-1,10-phenanthroline (1 g, 4.7 mmol) was placed in a 100 mL round bottomed flask equipped with a condenser and dissolved in 40 mL of DMF. NaH (0.6 g, 60% with mineral oil) was added and the slurry was stirred for 30 minutes at 25° C. before addition of 2-bromopropane (20 mL, about 200 mmol). The temperature was raised to 60° C. for 3 hours. The cooled reaction mixture was poured into 100 mL of water and extracted with 3×80 mL of CH₂Cl₂. The combined organic phases was dried with MgSO₄, concentrated in vacuo to yield a dark red solid. It was washed with 5×20 mL of hexane to remove the residual mineral oil and dissolved in 20 mL of dichloromethane and washed with 20 mL of 2M NaOH. The organic layer was separated, dried with Na₂SO₄ and the solvent evaporated under vacuum to yield a red solid of 750 mg (yield: 54%). ¹H-nmr(200 MHz, CDCl₃): δ 8.95(d, 5.29 Hz, 2H), 8.15(s, 2H), 6.95(d, 5.38 Hz, 2H), 4.85(heptet, 6.17 Hz, 2H), 1.5(d, 6.03 Hz, 12H). ¹³C-nmr(200 MHz, CDCl₃): δ 161.06, 151.42, 147.78, 122.01, 119.45, 104.49, 71.45, 22.33. MS/EI: M⁺=296, M⁺−2(C₃H₆)=212(base peak). MS/CI: M⁺+H=297(base peak).

### Preparation of cis-dichlorobis(N,N-dimethylamino)-1,10-phenanthroline} rhodium(III) chloride (BISNMe2)

100 mg of RhCl₃.3H₂O (about 0.38 mmol) was dissolved in 20 mL of distilled water and boiled. 200 mg of 4,7-bis(N,N-dimethylamino)-1,10-phenanthroline (0.75 mmol) was dissolved in 20 mL methanol and boiled. The hot boiling methanolic solution of the ligand was added carefully to the well stirred hot aqueous solution of RhCl₃. The resulting mixture was boiled for another 10 minutes. After cooling the mixture, a catalytic amount of NH₂NH₂.HCl was added to the mixture and boiled for another 15 minutes. Cooling the mixture to room temperature followed by further cooling to 5° C. gave orange crystals of bisNMe₂. The solid was filtered and washed with 3×50 mL of ice-cold water, followed by 3×10 mL of ice-cold methanol. The crystals were air dried, then dried under vacuum to give 289 mg of bisNMe₂ (yield: 96%). ¹H-nmr(dmso-d₆): δ 9.35(d, J=6.59 Hz, 2H), 8.22(d, J=9.57 Hz, 2H), 8.10(d, J=9.52 Hz, 2H), 7.50(d, J=6.72 Hz, 2H), 7.22(d, J=6.59 Hz, 2H), 6.75(d, J=6.88 Hz, 2H), 3.45(s, 12H), 3.20(s, 12H). LRMS (ESI) for positive ion 705/707. HRMS(ESI) for positive ion 705.1495 (Calculated), 705.1497 (Actual).

### Preparation of cis-dichlorobis(4,7-dimethoxy-1,10-phenanthroline) rhodium(III) chloride (TMOBP)

110 mg (0.43 mmol) of RhCl₃.3H₂O was dissolved in 20 mL of water by boiling. 25 mg (0.87 mmol) of 4,7-dimethoxy-1,10-phenanthroline (Aldrich Chemical Company) was dissolved in 20 mL by boiling. The methanolic solution of the ligand was added to the well stirred hot aqueous solution of RhCl₃ dropwise. The resulting mixture was boiled for 10 minutes. After cooling the mixture, 10 mg of NH₂NH₂.HCl was added to the mixture and boiled for another 15 minutes. Cooling the mixture to room temperature followed by further cooling to 5° C. gave yellow crystals of TMOBP. The solid was filtered and washed with 3×50 mL of ice-cold water, followed by 3×10 mL of ice-cold methanol. The crystals were air-dried, then dried under vacuum to give 301 mg (yield: 94%) of product. ¹H-nmr(200 MHz, DMSO): δ 9.8(d, 6.35 Hz, 2H), 8.5(d, 9.16 Hz, 2H), 8.35(d, 9.16 Hz, 2H), 8.10(d, 6.68 Hz, 2H), 7.85(d, 6.43 Hz, 2H), 7.20(d, 6.6 Hz, 2H), 4.45(s, 6H), 4.15(s, 6H). UV/Visible (in water): λₘₐₓ=348(7718 molL⁻¹cm⁻¹), 325(13500 molL⁻¹cm⁻¹), 313(13670 molL⁻¹cm⁻¹). MS/ESI: M⁺=653/655 for cation.

### Preparation of cis-dichlorobis(3,7-diisopropoxy-1,10 phenanthroline) rhodium(III) chloride (TIOBP)

166 mg of RhCl₃.3H₂O (0.63 mmol) was dissolved in 15 mL of distilled water and boiled. 375 mg of 4,7-diisopropoxy-1,10-phenanthroline (1.27 mmol) was dissolved in 15 mL of methanol and boiled. The hot methanolic solution of the ligand was added carefully to the well stirred hot aqueous solution of RhCl₃. The resulting mixture was boiled for another 10 minutes. After cooling the mixture to room temperature, a catalytic amount of NH₂NH₂.HCl was added to the mixture and boiled fro another 15 minutes. Cooling the mixture to room temperature followed by further cooling to 5° C. gave yellow crystals of TIOBP. The solid was filtered and washed with 3×25 mL of ice-cold water, followed by 3×10 mL of ice-cold methanol. The crystals were air dried, then dried under vacuum to give 390 mg of TIOBP (yield: 73%). ¹H-nmr(dmso-d₆): δ 9.65(d, J=6.3 Hz, 2H), 8.4(d, J=9.15 Hz, 2H), 8.25(d, J=9.15 Hz, 2H), 8.05(d, J=6.88 Hz, 2H), 7.75(d, J=6.59 Hz, 2H), 7.2(d, J=6.68 Hz, 2H), 5.3(septet, J=6.18 Hz, 2H), 4.95(septet, J=6.15 Hz, 2H), 1.6(triplet, J=5.3 Hz, 12H), 1.4(dd, J=14.5 Hz, 5.9 Hz, 12H). LRMS (ESI) for positive ion 765/767. HRMS(ESI) for positive ion 765.1483 (Calculated), 765.1501 (Actual).

### Example VII

### Synthesis of cis-dichlorobis(4,7-diphenyl-1,10-phenanthroline) rhodium(III) chloride (TPBP)

123 mg of RhCl₃.3H₂O (about 0.5 mmol) was dissolved in 10 mL of distilled water and boiled. 548 mg (about 1.65 mmol) of 4,7-diphenyl-1,10-phenanthroline was dissolved in 20 mL of 95% ethanol. The hot ethanolic solution of the ligand was added carefully to the well-stirred hot aqueous solution of RhCl₃. The resulting mixture was boiled for another 30 minutes, adding more distilled water to keep the volume of the solution about 20 mL. Upon cooling the mixture, a yellow solid crystallized out. The solid was filtered out and washed with ice-cold water and with ice-cold ethanol. The crystals were air-dried, then dried under vacuum to give 630 mg of TPBP. ¹H-nmr(cdcl₃): δ 10.07(d, J=5.4 Hz, 1H), 8.18(d, J=5.4 Hz, 1H), 8.00(d, J=90 Hz, 1H), 7.94(d, J=5.94 Hz, 1H), 7.88(d, J=9.6 Hz, 1H), 7.57-7.53 (two doublets, J=7.2 Hz, 5.4 Hz, 3H), 7.39-7.44 (m, 3H). LRMS (ESI) for positive ion 837/739. HRMS(ESI) for positive ion 837.1059 (Calculated), 837.1022 (Actual).

### Example VIII

### Bioactivity of DPPZPHEN: Photonicking and Phototoxicity

DPPZPHEN (Example I, FIG. 1c), also known as cis-Rh(dppz)(phen)Cl₂⁺, is an octahedral rhodium compound having the chemical formula cis-dichloro(dipyrido [3,2-a:2′,3′-c]phenazine)(1,10-phenanthroline) rhodium(III). DPPZPHEN is an analog of BISPHEN in which one of the phen ligands has been replaced by the dipyrido[3,2a-2′3′c]phenanzine (dppz) moiety. The dppz ligand is one of the most used polypyridyl ligands in metal complexes for studies of interactions with DNA (Waterland et al., J. Raman Spectrosc., 31:243-253 (1999)). Metal complexes containing a dppz ligand bind strongly to DNA via intercalation of the dppz structural motif. The binding constants of the metal complexes containing dppz are on the order of 10⁶ to 10⁷ M⁻¹ for dicationic metal complexes, and 10⁴ to 10⁵ M⁻¹ for monocationic metal complexes. (Stoeffer et al., J. Am. Chem. Soc., 117:7119-7128 (1995)). The dppz ligand was selected to give the metal complex intermediate hydrophobicity while retaining the stability of BISPHEN and the intercalating capability of PHENPHI.

Among recent literature reports of metal complexes using the dppz ligand for the development of new diagnostic and therapeutic agents that can recognize, covalently bind to, and/or cleave DNA, are complexes using Co (Arounaguiri et al., Inorg. Chem., 35:4267-4270 (1996)), Ni (Arounaguiri et al., Inorg. Chem., 35:4267-4270 (1996)), Os (Holmlin et al., 34:7-8 (1995); Holmlin et al., Inorg. Chem., 38:174-189 (1999)), Re (Stoeffer et al., J. Am. Chem. Soc., 117:7119-7128 (1995); Yam et al., J. Chem. Soc. Dalton Trans., 12:2067-2072 (1997)), Cu (Waterland et al., J. Raman Spectrosc., 31:243-253 (1999)), and Ru (Arounaguiri et al., Inorg. Chem. 38:842-843 (1999); Sook et al., Biopol. 67:121-128 (2002); Chen et al., J. Am. Chem. Soc. 124:3064-3082 (2002)). Some of these metal complexes were found to be able to photonick DNA (Arounaguiri et al., Inorg. Chem., 35:4267-4270 (1996); Yam et al. J. Chem. Soc. Dalton Trans., 12:2067-2072 (1997)). However, there appear to have been no reports of a dppz rhodium complex as a therapeutic agent.

The following abbreviations are used in this example: ROS, reactive oxygen species; SINV, Sindbis virus; KB, human nasopharingeal tumor cells; M109, human lung tumor cells; GN4, canine prostate tumor cells; BHK, baby hamster kidney cell; CT DNA, calf thymus DNA; BISPHEN, cis-dichlorobis(1,10-phenanathroline) rhodium(III) chloride; DPPZPHEN, cis-dichloro(dipyrido[3,2a-2′3′c]phenazine)(1,10-phenantroline) rhodium(III) chloride; min, minute(s); h, hour(s).

Materials and Methods

General. The quantity of DNA was determined by the Burton assay (Burton, Biochem. J., 62:315-322 (1956)), and its rhodium content was quantitated using flame atomic absorption spectroscopy (AA). The equilibrium constant for interaction between DPPZPHEN and CT-DNA in the dark was calculated from UV-Vis absorption titration measurements at 380 nm (Wolfe et al., Biochem., 26:6392-6396 (1987); Schemel et al., Biopolymers, 10:465-480 (1971)). Cell viability was determined using the MTT assay (Denizot et al., J. Immunol. Methods, 89:271-277 (1986)). SINV was purified on sucrose gradients as previously described (Owen et al., J. Virol., 70:2757-2763 (1996)). The titer was generally 7-8 log₁₀ pfu/mL at the beginning of each experiment.

Photolysis with Calf Thymus DNA (CT DNA). Solutions consisting of 1 mL aliquots of 0.58 mM DPPZPHEN and 2.7 mg/mL CT DNA were irradiated with λ>330 nm light. After irradiation, the solutions were purified by exhaustive dialysis and three cycles of precipitation and resuspension. The DNA and Rh content were determined and/or the DNA was utilized in size exclusion chromatography experiments (Mahnken et al., J. Am. Chem. Soc., 114:9253-9265 (1992)).

Photolysis with φX-174 Plasmid DNA. Metal complex solutions (with or without quenchers) were saturated with oxygen or argon prior to irradiation. The solutions were further degassed after being mixed with the plasmid. The final solution concentrations were: DPPZPHEN (12), φX-174 plasmid DNA (205 μM in base pairs), superoxide dismutase (25 μg/mL), 2-propanol (0.5 M), mannitol (6.25 mM), histidine (8 mM). The samples were irradiated with 311 nm light and the solutions subjected to gel electrophoresis.

Photolysis with Tumor Cells. KB, GN4 and M109 cells were plated substantially as described in Example X, below. At the beginning of an experiment media was removed from all plates and the cells were washed with 1 mL of PBS or Hank's salt solution. Drug solution (2 mL) or buffer solution (2 mL) was added to each dish. Control solutions did not contain the metal complex. Cells were incubated, with or without metal complex, for 4 hours. After incubation, the solutions were removed from the dishes and the dishes were washed with 1 mL of PBS or Hank's salt solution. A fresh 1 mL of PBS or Hank's salt solution was added to each dish and these were placed on a turntable that sat on the top part of the photolysis chamber. The dishes were irradiated from below with 311 nm light, the drug or control solutions removed from the dishes, and 2 mL of fresh media was added to each dish. The plates were then incubated for 40-48 hours before a determination of the extent of cell toxicity by MTT assay.

Photolysis with SINV. DPPZPHEN/SINV solutions were prepared by adding a 0.3 mL aliquot of a 600 μM DPPZPHEN stock solution in PBS to 3 mL of purified virus at a concentration of 7-8 log₁₀ pfu/mL and bringing the total volume to 4 mL. The DPPZPHEN/SINV solutions were incubated for 2 hours prior to irradiation, following which 3 mL of the solution was irradiated in quartz cuvettes with 355 nm light under a constant stream of nitrogen with stirring Aliquots of 100 μL were withdrawn over time and analyzed for virus infectivity. The inactivation rate constant was calculated using simple hit theory (Hiatt, Bacteriol. Rev., 28:150-163 (1964); Houghtaling et al., Photochem. Photobiol., 71:20-28 (2000)). For the studies measuring infectivity of genome RNA, a 6.9 μL aliquot of 0.623 M DPPZPHEN stock solution in DMSO was added to a 100 μL of TE12 wild type SHIN solution (10 log₁₀ pfu/mL) to obtain a 40 mM DPPZPHEN/virus solution. Aliquots of 50 μL were placed into 250 μL inserts, incubated for 2 hours, and either kept in the dark or irradiated for 60 minutes with light >330 nm. At the completion of the experiment all the samples were diluted with 6 mL 1×PBS buffer and precipitated for 2 hours at 4° C. with 10% PEG+NaCl (200 mM). The PEG precipitated virus was centrifuged at 4° C. for 30 minutes at 14,000 rpm, and the resulting pellet re-suspended in 200 μL of 1×PBS buffer and extracted with equal volumes of phenol and chloroform. The virion RNA was precipitated with ethanol, re-suspended in 100 μL of 1×PBS, and electroporated into about 2×10⁷ baby hamster kidney (BHK) cells using 0.2-cm-gap cuvettes. The electroporation utilized 200 Ohms, 1.5 kV, and 25 μF. The cells were incubated at 37° C. for 12 hours in MEM supplemented with 10% fetal calf serum and the viral supernatants were harvested and assayed for the presence of infectious virus by plaque assay.

An immunofluorescence assay was also employed to detect the presence of SINV capsid protein newly translated from the irradiated viral genomic RNA. For these experiments the virus solutions had to be 10 log₁₀ pfu/mL, which is a virus concentration 3 orders of magnitude higher than that used in the previous experiments. Also, the DPPZPHEN solutions had to be prepared in DMSO to maintain the ratio of drug to virus as in the previous experiments. Preliminary results demonstrated that solutions with up to 10% DMSO v/v had no effect on the virus or on the rate of photoinactivation by DPPZPHEN. SINV RNA from virus that had been either incubated in the dark or irradiated with DPPZPHEN was isolated and transfected into baby hamster kidney cells as described above. Following electroporation, the cells were plated on coverslips and incubated at 37° C. for 12 hours. The cells on the coverslips were then fixed with methanol for 15 minutes and washed twice with 1×PBS buffer. They were then incubated with anti-CP (SINV) polyclonal antibody (Owen et al., J. Virol., 70:2757-2763 (1996)) for 45 minutes at 37° C. After washing three times with 1×PBS buffer the cells were incubated with fluorescein-5-isothiocyanate (FITC) conjugated goat anti-rabbit secondary antibody (obtained from Kirkegaard and Perry, Gaithsburg, Md.) at 37° C. for 45 minutes. The control and experimental samples were also stained with DAPI (4,6-diamidino-2-phenylindole), which was used as a counter stain to highlight the nuclei of BHK cells. The coverslips were washed three times with 1×PBS and mounted on microscope slides using Fluorosave Reagent (Calbiochem, La Jolla, Calif.). They were viewed under a Nikon Eclipse TE200 with a TE-FM Epi-fluorescence attachment.

Results

Interaction of DPPZPHEN with DNA. Given the results that had been obtained with the DPPZPHEN precursor, BISPHEN (Billadeau et al., Metal Ions Biol. Syst., 33:269-296 (1996); Mahnken et al., J. Am. Chem. Soc., 114:9253-9265 (1992); Billadeau et al., Inorg. Chem., 33:5780-5784 (1994); Harmon et al., Inorg. Chem., 34:4937-4938 (1995); Mohammad et al., Biorg. Med. Chem. Lett., 9:1703-1708 (1995); Morrison et al., Photochem. Photobiol., 72:731-738 (2000)), we expected to observe significant interaction of the metal complex with nucleic acid, both in the dark and upon irradiation. Titration of a DPPZPHEN solution with CT DNA caused a small bathochromic shift and hypochromism of the near-UV dppz absorption spectral features with increasing amounts of DNA. These results are consistent with intercalation of the complex into the DNA (Bloomfield et al., Physical Chemistry of Nucleic Acids, Harper and Row, New York, page 432), as has been observed with other dppz transition metal complexes (Stoeffer et al., J. Am. Chem. Soc., 117:7119-7128 (1995); Yam et al., J. Chem. Soc. Dalton Trans., 12:2067-2072 (1997); Long et ale, Acc. Chem. Res., 23:271-273 (1990); Wilhemsson et al., J. Am. Chem. Soc., 124:12092-12093 (2002)). An intrinsic binding constant of 1% 3.4×10⁵ M⁻¹ was calculated using the molar extinction coefficient at the λₘₐₓ (380 nm) at each titration point. In contrast, BISPHEN showed negligible interaction with CT DNA in the dark.

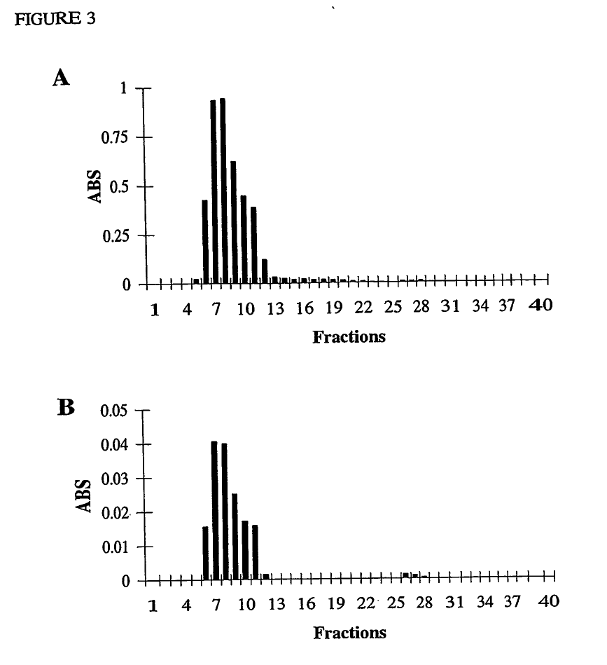

Irradiation of the CT DNA-DPPZPHEN complex with >330 nm light, under argon or oxygen, led to the recovery of nucleic acid containing 165 and 155 mmol of rhodium bound per mg DNA under argon and oxygen, respectively. These levels correspond to about 1 molecule of Rh per 18 bases. Size exclusion chromatography was used to confirm that the metal was indeed covalently bound to the DNA. The complex and the nucleic acid co-eluted at a volume between 18 to 36 mL (fractions 6 to 13) from a 40 mL wet bed Sephadex column (as evidenced by concomitant analysis of eluents at 260 nm (FIG. 3a) and 380 nm (FIG. 3b). A control run with DNA mixed with the complex in the dark showed the complex eluting much later in the chromatogram (between 78 to 93 mL data not shown), well resolved from the DNA. These results are consistent with the extensive studies of BISPHEN with CT and plasmid DNA that showed the rhodium complex to be preferentially photolytically bound to purines, particularly strings of guanines.

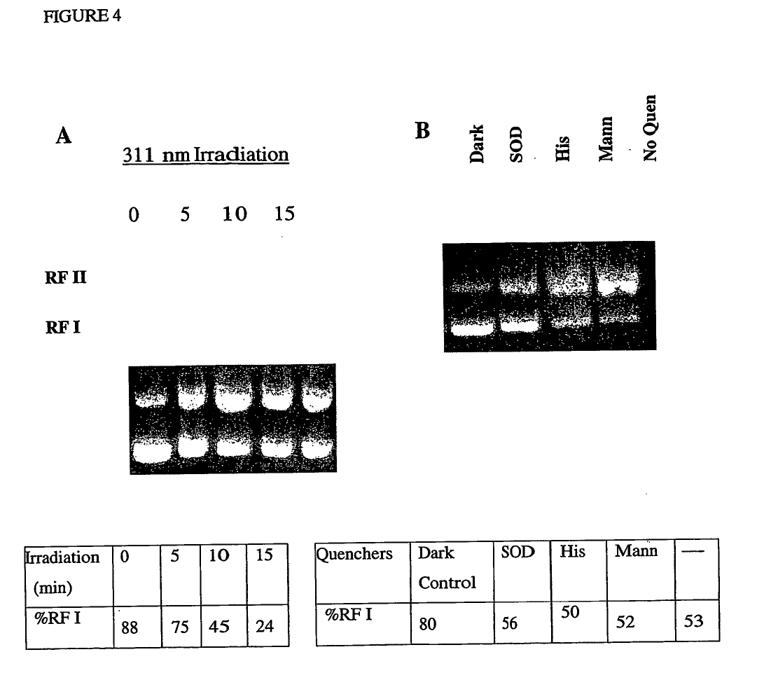

Though the covalent binding of metal to the nucleic acid was well documented in our earlier studies, the observation of frank nicks upon irradiation of φX-174 supercoiled plasmid DNA with DPPZPHEN was not. Nicking of the plasmid was found to be light dose dependent, as seen in FIG. 4. As shown in an analogous study, BISPHEN does not generate such frank nicks. Other studies have also shown that metal complexes containing the dppz ligand nick DNA when irradiated but no mechanism has yet been described for this phenomenon (Stoeffer et al., J. Am. Chem. Soc., 117:7119-7128 (1995); Arounaguiri et al., Inorg. Chem., 35:4267-4270 (1996)). It is evident, however, that diffusible reactive oxygen species do not seem to be required. When φX-174 plasmid DNA was irradiated with DPPZPHEN in oxygen-saturated solutions in the presence of various quenchers (FIG. 4), and under argon, there was no significant level of protection of the DNA observed relative to a simple buffered control.

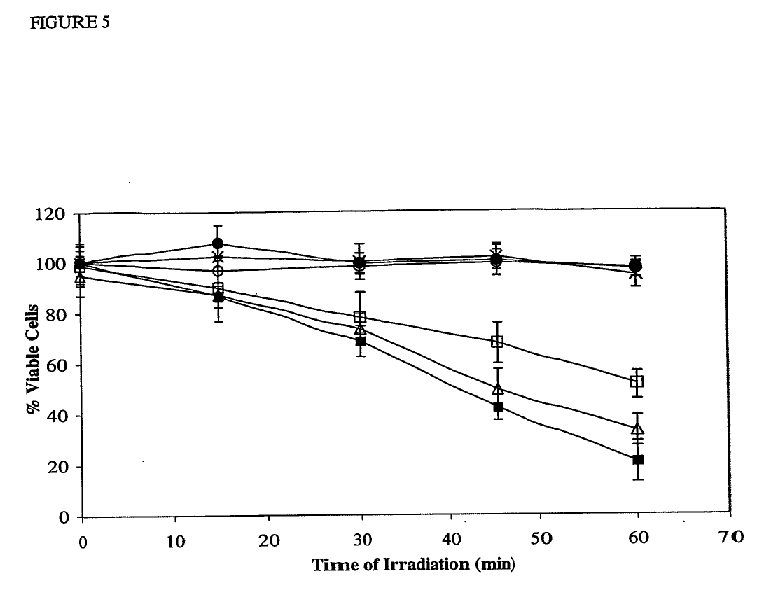

Tumor Cell Phototoxicity. The results of the irradiation of DPPZPHEN with the GN4, M109 and KB tumor cell lines, using 311 nm light, are presented in FIG. 5. As much as 80% of the KB cells were rendered nonviable in 60 minutes The complex was slightly less effective on the M109 line and least effective on (about 50% toxicity) GN4 cells. There was no appreciable degree of cell toxicity in the absence of light (zero time point in FIG. 5), nor were any of the cell lines affected by light in the absence of DPPZPHEN. The phototoxicity was dose dependent for all three cell-lines, with the level of toxicity relatively constant between 10 and 40 μM metal complex. This suggests that the three cell lines are saturated at a common intracellular concentration of the metal complex (data not shown).

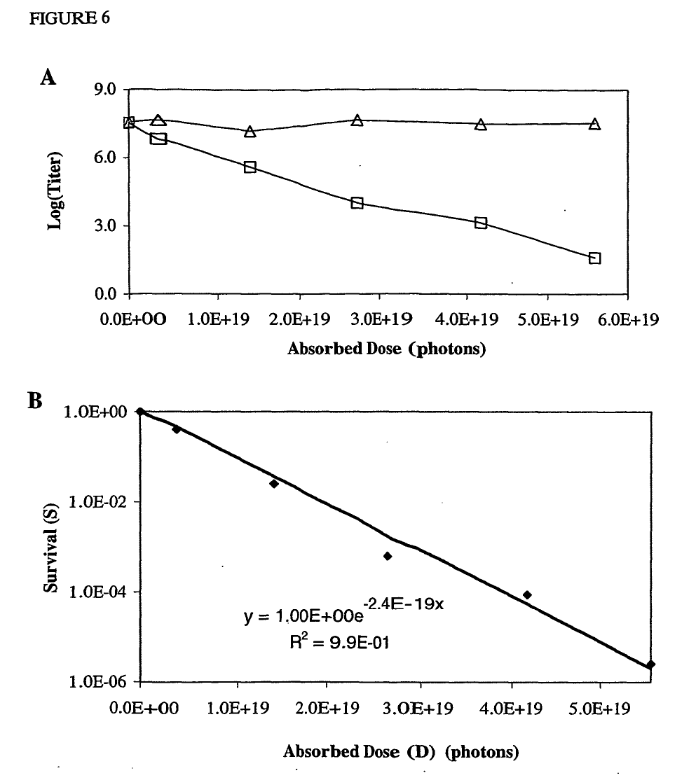

Photoinactivation of SINV. FIG. 6A shows the effect of 45 μM DPPZPHEN on SINV. The complex proved to be quite potent towards the virus, with a 6 log reduction in viral titer over the course of the experiment. Simple hit theory analysis of the data provides an inactivation rate constant k=2.4×10⁻¹⁹ photons⁻¹ (FIG. 6B). The virus was unaffected by irradiation in the absence of the metal complex or by the metal complex in the dark (FIG. 6A). Additionally, the rate of photoinactivation was the same under either oxygen or nitrogen. These observations are consistent with the model studies with CT and φX174 plasmid DNA and confirm that the metal complex is indeed photoactive in a hypoxic environment.

Confirmation of RNA as the Viral Target. Since the SINV genomic RNA is infectious, RNA isolated from virions can be used to probe whether DPPZPHEN directly targets the viral genome. For this purpose, RNA from virus that had been irradiated in the presence of the metal complex was isolated and transfected into susceptible cells to investigate if DPPZPHEN inactivated SINV by damaging its viral genome. Virus irradiated without the metal complex and a DPPZPHEN/SINV solution kept in the dark were used as controls Cells that were transfected with RNA from these controls showed a complete cytopathic effect in 48 hours post transfection. However, cells that received RNA isolated from virus irradiated with DPPZPHEN showed no plaque formation. An immunofluorescence assay was utilized to confirm that the DPPZPHEN photodamage resulted in SINV RNA inactivation. RNA isolated from virus irradiated with or without DPPZPHEN was transfected into BHK cells as previously described, and the presence of newly translated SINV CP was analyzed by immunofluorescence assays. Since SINV replicates in the cytoplasm of BHK cells, the CP was expected to localize in the cytoplasm. CP was not observed in cells transfected with RNA isolated from virus irradiated with DPPZPHEN. However, CP was observed in cells transfected with the control RNA. DAPI was used as a counter stain to highlight the nuclei of individual BHK cells. It must be noted, however, that a low level of transfected cells was observed in these experiments due the difficulty of isolating sufficient amounts of RNA from purified virions.

Conclusions

We have demonstrated that a new octahedral rhodium complex, DPPZPHEN, shows phototoxicity towards tumor cells and enveloped animal viral particles. The complex is fairly effective against KB and M109 cell lines, with about 80% cell death in 60 minutes of 311 nm irradiation. It has a more modest effect on GN4 cells, causing 50% cell death under comparable conditions. The complex is appreciably more effective against SINV, reducing viral viability by 6 log, with a rate of inactivation of k=2.4×10⁻¹⁹ photons⁻¹. We have also shown that the SINV genome is a primary target for the complex. This observation is significant. As a member of the Togaviridae family, SINV has a complex structure consisting of an outer glycoprotein shell, a lipid bilayer and an inner nucleocapsid shell that surrounds the single strand RNA genome. Therefore, the ability of this metal complex to directly target the viral genome indicates that it is capable of penetrating two protein layers and the lipid bilayer. Furthermore, by producing an extensive amount of damage to the pathogen genome, DPPZPHEN “kills” the infectious agent rather than inhibiting one of its biological functions, thus curbing the pathogen's ability to develop resistance. The ability to “kill” pathogens rather than inhibit their biological functions is thought to be of paramount importance for the effectiveness of photoactive drugs. A similar observation has been reported for methylene blue, which is a photoactive drug known to “kill” methicillin or vancomycin resistant strains of Staphylococcus aureus (Wainwright et al., FEMS Microbiol. Lett., 160:177-181 (1998); Wainwright et al. (1999) Antimicrob. Chemother., 44:823-825 (1999)).

Since DPPZPHEN has demonstrated a dual mode of reaction with nucleic acid, i.e. covalent binding as well as nicking, it is not possible to provide specific details for a mechanism of viral and cellular toxicity. It is noteworthy, however, that neither photonicking of, nor photobinding to, nucleic acid was found to be affected by the presence of oxygen, thus eliminating the involvement of diffusible reactive oxygen species in the phototoxic sequence These observations, and the lack of a dependence of DPPZPHEN's viral phototoxicity in the presence of oxygen, suggest the possibility that such complexes and their analogs might be effective against hypoxic tumor cells. Additionally, DPPZPHEN's lack of production of ROS, its high association constant with DNA (K_{eq}=3.4×10⁵ M⁻¹), and its ability to target the viral genome, are all attractive characteristics for the ultimate design of phototoxic agents that might be free of indiscriminate photosensitized damage of blood components. The latter represents one of the major drawbacks of currently employed photodecontaminating drugs (Santus et al., Clin. Hemorheology and Microcirculation, 18:299-308 (1998)) that rely on the formation of singlet oxygen for their effectiveness (Wainwright, Current Med. Chem., 9:127-143 (2002)).

In summary, these results provide a proof of principle for the use of rhodium metal complexes as phototoxic agents that appear to have a universal targeting mechanism for both the DNA and RNA genomes. With the ongoing discovery of new infectious agents transmitted through blood products, the further development of DPPZPHEN analogs may lead to efficacious and cost-effective strategies for securing the blood supply against unforeseen pathogens, since the use of such compounds does not require prior knowledge of the presence of infectious agents.

### Example IX

### Photonicking of φX-174 Plasmid DNA by Various Rh Bipyridyl Metal Complexes

Experiments were conducted as described in Example X to evaluate the amount photonicking that occurred in the dark and upon exposure to light for 37TMBP, 56TMBP, OCTBP, BISNMe2, TMOBP, TIOBP, TPBP, BISTAP and BISDPPZ. Only TPBP, BISTAP, BISDPPZ and BISDPPHEN complexes were found to photonick the φX-174 plasmid DNA.

### Example X

### Phototoxicity Against Tumor Cells by Octamethylbisphen (OCTBP)

OCTBP uptake by KB cells. 11 mg of OCTBP was dissolved in 40 mL of PBS buffer. The solution was vortex stirred for 5 minutes to completely dissolve the solid. The solution was sterile filtered through a 0.2μ nylon syringe filter. The concentration of the solution was determined by measuring the UV absorption at 353 nm (ε₃₅₃ₙₘ=1565 M⁻¹cm⁻¹). The concentration of the solution was 33 μM.

Once the cells were 75% confluent media was decanted from the flasks and 8 mL of drug solutions were added to three flasks and 8 mL of PBS buffer solution was added to the control flask. The flasks were incubated at 37° C. for 15, 30 and 45 minutes. Control flask was incubated for 45 minutes. When the flasks were removed, they were washed with 6×10 mL of PBS buffer solution. All the liquid was carefully decanted from the flasks. 1 mL of lysing buffer (1% triton X-100) was added to each of the four flasks. The walls of the flasks were rinsed using disposable pipettes. Once all the contents of the flasks were placed into a 1.5 mL disposable centrifuge tubes (each flask yielded ˜1.2 mL), the tubes were centrifuged at 6000 rpm and at 4° C. for 30 minutes on the Fisher Model 59V high-speed centrifuge. BCA Assay was done to establish the amount of cells in each flask.

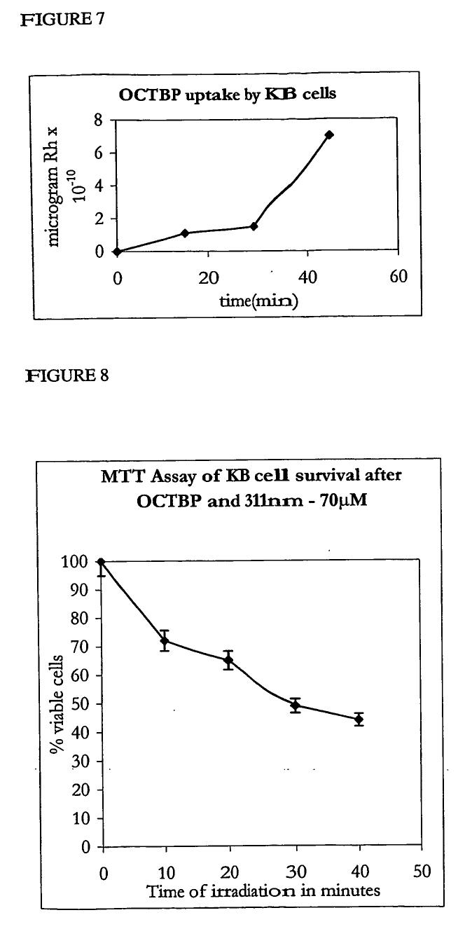

Rh content of each flask was determined using an inductively coupled plasma (ICP) instrument (AtomScan16, Thermo Elemental, Franklin, Mass.). From each centrifuge tube 1 mL was pipetted and 370 μL of concentrated HCl was added to form a 10% solution. The samples were heated in a hot water bath at 70° C. for 30 minutes. The samples were centrifuged again and the supernatant liquid was diluted to 8 mL by adding nano-pure water. The samples were analyzed for Rh content by ICP. OCTBP uptake is shown in FIG. 7.

MTT Assay of KB cell line survival after treatment of OCTBP and UVA (311 nm) irradiation (70-micromolar solutions). Cells were incubated in 75 cm² culture flasks. They were grown in Dulbecco's Modified Eagles Medium (D-MEM) supplemented with 10% heat inactivated fetal bovine serum (FBS), 1% L-glutamine-penicillin-streptomycin and 1% amphotericin-B. When cells were not plated for use in an experiment, they were transferred when they were 75-100% confluent using the following procedure. Media was decanted from the flask. Cells were rinsed 1-2 times with 10 mL of Hank's Balanced salt solution. 1 mL of Trypsin-EDTA (0.5% Trypsin.5.3 mM EDTA.4Na—Life Technologies cat # 15400-054) was added to the flask and the cells were left in contact with this solution for 1 minute. Trypsin-EDTA was then decanted and the cells were released from the wall of the flask by tapping the side of the flask. Released cells were suspended in 5 mL media and 1 mL of this suspension was placed in a new culture flasks containing 20 mL of fresh media. The remaining cells were discarded. A culture flask can be used for 3 transfers.

Usually 16-24 hours before a photolysis experiment, cells were plated in 35×10 mm tissue culture dishes and incubated at 37° C. until the experiment was begun. Cells were about 75-100% confluent in the 75 cm² culture flask when plated. After being washed and released from the flask as described above, cells were suspended in 40 mL media and 1.5 mL of suspension was placed in each dish.

20 mg of OCTBP was dissolved in 20 mL of sterile Hank's balanced salt solution by vigorously vortex stirring for 15 minutes. The solution was filtered through a sterile 0.2μ nylon syringe filter and the concentration of the filtrate was determined by UV measurement at 312 nm. The concentration of the solution was 70 μM.

The cell dishes were incubated with drug or Hanks balanced salt solution (1 mL) for 3 hours. After incubation, solutions were decanted from the plates and 1 mL of Hank's Balanced salt solution were placed in each. Cells were then photolyzed by 311 nm lamps (2 lamps) for 10, 20, 30 and 40 minutes. Following photolysis, Hank's Balanced salt solutions were decanted and 1 mL of fresh media was placed in each plate.

Plates were incubated for 65 hours and then analyzed for cell survival by MT assay. 5 mg/mL MTT solution in water was prepared and 100 μL of this solution was added to each plate and the plates were incubated for 1.5 hours. Purple crystals formed from live cells. Media was gently removed from plates using a Pasteur pipette, the purple crystals that remained at the bottom of the dish were dissolved in 6 mL of isopropanol. The absorbances of these solutions at 570 nm were measured. The absorbance are indicative of relative cell survival, as shown in FIG. 8.

Determination of the optimal concentration of OCTBP for the phototoxic to KB cell lines at 311 nm. The above-mentioned procedure was repeated with various concentrations of OCTBP solutions to induce a maximum phototoxicity to KB cell lines with a minimum amount of the drug. The optimal concentration of OCTBP to create considerable amount of photosensitized death in KB cell lines was found to lie between 55 μM to 70 μM. When the concentration was increased to 80 μM considerable amount of dark toxicity also was observed. At 70 μM solution dark toxicity wasn't observed. To minimize the dark toxicity, 55 μM solution can be used.

MTT Assay of GN4 cell survival after treatment of OCTBP and UVA (311 nm) irradiation Cells were incubated in 75 cm² culture flasks. They were grown in Minimum Essential Medium (MEM) supplemented with 5% heat inactivated fetal bovine serum (FBS), 1% L-glutamine-penicillin-streptomycin, 1% amphotericin-B and 1% (v/v) MEM non essential amino acid solution. When cells were not plated for use in an experiment, they were transferred when they were 75-100% confluent using the following procedure. Media was decanted from the flask. Cells were rinsed 1-2 times with 10 mL of Hank's Balanced salt solution. 2 mL of Trypsin-EDTA (0.5% Trypsin 5.3 mM EDTA.4Na—Life Technologies cat # 15400-054) was added to the flask and cells were left in contact with this solution for 8 minutes. Trypsin-EDTA was then decanted and the flask was incubated at 37° C. for 8 minutes. The cells were released from the wall of the flask by tapping the side of the flask. Released cells were suspended in 5 mL of media and shaken well. 1 mL of this suspension was placed in a new culture flask containing 10 mL of fresh media. The remaining cells were discarded. A culture flask can be used for 3 transfers.

Cells were plated, incubated with the drug, and photolysis was conducted as for the KB cell lines.

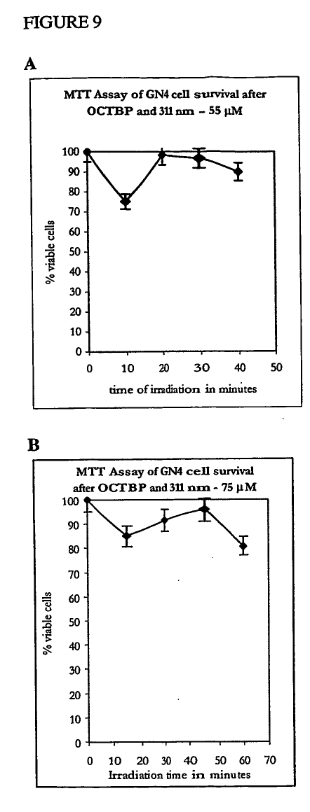

It was found that no photo-cell death occurred in GN4 cell lines at 55 μM concentration (FIG. 9a), which is an optimal concentration to create considerable photo initiated cell death in KB cell lines. Thus GN4 cell lines were more resistant to OCTBP than KB cell lines. When a 75 μM solution was used a small amount of photo-initiated cell death occurred in GN4 cell lines (about 20% in 40 minutes) (FIG. 9b). A longer period of incubation of the drug and/or longer period of irradiation may increase the phototoxicity in the GN4 cell lines.

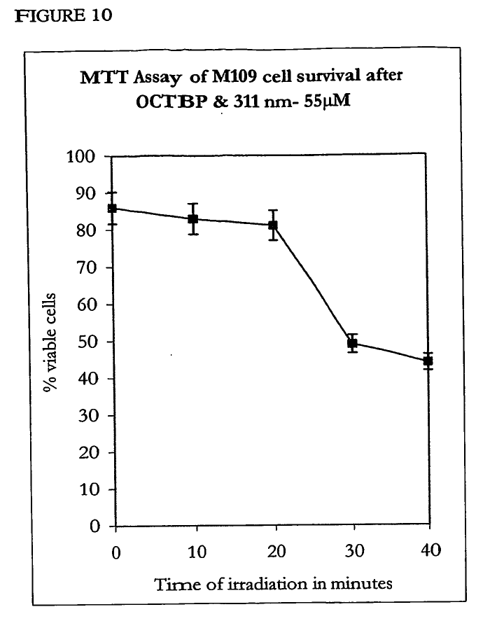

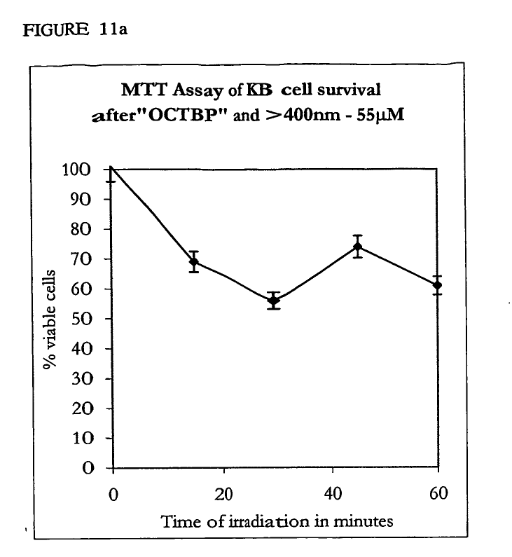

MTT Assay of M109 cell survival after treatment OCTBP and UVA (311 nm) irradiation. Procedures for cell maintenance, cell plating, drug incubation, photolysis and MTT assay are all same as that used for the KB cell lines. A small amount of dark toxicity was observed (about 15%). A considerable amount of killing occurs with light alone after 30 and 40 minutes of irradiation (FIG. 10).

Phototoxicity studies with long wavelength light (>400 nm). The same procedure was repeated and the cells were irradiated with a 500 W tungsten projector lamp filtered >400 nm by a long pass band filter. The photolysis was carried out in a turn-table (used for 311 nm photo box) to allow the same dosage of light to each cell dish.

Considerable amount of cell death occurred in KB cell lines (40% in 60 minutes in 55 μM solution) (FIG. 11a) whereas a small amount of cell death occurred in GN4 cell lines (25% in 60 minutes in 63 μM solution) (FIG. 11b). This may be due to the fact that GN4 cell lines are more resistant to OCTBP than KB cell lines.

### Example XI

### Dark Association of OCTBP, BISTAP and BISNMe2 with DNA

Preparation of DNA solution. Calf thymus DNA (Sigma) was dissolved in 50 mM, pH 7, I=0.15 sodium phosphate buffer (Physiological condition) and sonicated. The purity of the DNA was checked by monitoring the absorption spectrum and the ratio of the absorbance at 260 to 280 nm. The ratio were within the acceptable purity margin (1.8±0.1). The concentration of the Calf thymus DNA per phosphate was determined spectrophotometrically: ε₂₅₃=6600 for calf thymus DNA¹. The concentration was 2.13 E-3 M.

Titration of OCTBP with DNA. 1.0 mg of OCTBP was dissolved in 200 μL of dimethylsulfoxide (DMSO) and 14.8 mL of 50 mM, pH 7, I=0.15 sodium phosphate buffer (physiological condition) was added to it. The resulting solution was diluted 4 times with phosphate buffer. The concentration of the diluted solution was 2.26E-5 M. 2.3 mL of the diluted solution was pipetted into a UV-Visible cell and the absorption spectrum was taken. 0, 50 μL, 150 μL, 200 μL, 300 μL, 350 μL and 500 μL of DNA solutions were added, mixed well with a small disposable needle, and spectra were recorded again. The concentration of the species, the apparent absorbance at 337 nm, and the apparent extinction coefficients at 337 nm were determined from the absorptions spectra and tabulated in Table 1.

TABLE 1

| | | | | Δεₐₚₚ = | |
|---|---|---|---|---|---|
| [DNA] | [OCTBP] | A_{observed} | εₐₚₚ | ε_{f} − εₐₚₚ | [DNA]/Δεₐₚₚ |
| 0 | 2.26E−5 | 0.216 | 9558 | 0 | 0 |
| 4.53E−5 | 2.21E−5 | 0.214 | 9683 | 125 | 3.624E−7 |
| 1.30E−4 | 2.12E−5 | 0.209 | 9858 | 300 | 4.333E−7 |
| 1.70E−4 | 2.08E−5 | 0.206 | 9904 | 346 | 4.913E−7 |
| 2.46E−4 | 2.00E−5 | 0.199 | 9950 | 392 | 6.276E−7 |
| 2.81E−4 | 1.96E−5 | 0.195 | 9949 | 391 | 7.187E−7 |
| 3.80E−4 | 1.86E−5 | 0.187 | 10054 | 496 | 7.661E−7 |

The apparent binding constant, Kₐₚₚ was determined from the plot of [DNA]/Δεₐₚₚ versus [DNA], where Δεₐₚₚ=ε_{f}−εₐₚₚ and Δε=ε_{f}−ε_{b}², where ε_{b} and ε_{f} correspond to the extinction coefficient of the DNA-bound form of the OCTBP derivatives and the extinction coefficient of the DNA-unbound OCTBP derivatives, respectively. The apparent extinction coefficient, εₐₚₚ, was obtained by calculating A_{observed}/[OCTBP derivatives], where A_{observed} corresponds to the observed absorbance at absorption maxima. The data were fitted to Equation 1 wherein a slope equal to 1/Δε and a y-intercept equal to 1/[Kₐₚₚ·Δε] were obtained. ε_{b} was determined from Δε and Kₐₚₚ from the ratio of the slope to the y-intercept.
[DNA]/Δεₐₚₚ=[DNA]/Δε+1/Kₐₚₚ.Δε]
From the graph 1/Δε=0.0013, 1/[Δε.Kₐₚₚ]=3 E-7
Therefore Kₐₚₚ.=0.0013/3 E-7=4.25 E3 M
Δε=769.23
Therefore ε_{b}=ε_{f}−Δε=9558−769.23=8788.77
The concentration of the DNA-bound OCTBP (C_{b}) can be determined as follows (Chaires, Biochemistry, 21:3933-3940 (1982)).
C_{b}=(ε_{f}Cₜₒₜₐₗ−A_{observed})/Δε
The concentration of the DNA-unbound OCTBP derivatives (C_{f}) can be determined by
C_{f}=Cₜₒₜₐₗ−C_{b}

Titration of BISTAP with DNA. 1.8 mg of BISTAP was dissolved in 200 μL of DMSO and 9.8 mL of 50 mM, pH 7, I=0.15 sodium phosphate buffer (physiological condition) was added to it. The resulting solution was diluted 10 times with phosphate buffer. The concentration of the diluted solution was 2.48 E-5 M. 2.3 mL of the diluted solution was pipetted into a UV-Visible cell and the absorption spectrum was taken. 0, 50 μL, 150 μL, 200 μL, 250 μL, 350 μL and 400 μL of DNA solutions were added, mixed well with a small disposable needle and spectra were recorded again. The concentration of the species, the apparent absorbance at 344 nm, the apparent extinction coefficients at 344 nm were determined from the absorptions spectra and tabulated in Table 2.

TABLE 2

| | | | | Δεₐₚₚ = | |
|---|---|---|---|---|---|
| [DNA] | [BISTAP] | A_{observed} | εₐₚₚ | ε_{f} − εₐₚₚ | [DNA]/Δεₐₚₚ |
| 0 | 2.48E−5 | 0.2340 | 9435 | 0 | 0 |
| 4.53E−5 | 2.43E−5 | 0.2265 | 9321 | 114 | 3.97E−7 |
| 1.30E−4 | 2.33E−5 | 0.2146 | 9210 | 225 | 5.78E−7 |
| 1.70E−4 | 2.28E−5 | 0.2109 | 9250 | 185 | 9.19E−7 |
| 2.09E−4 | 2.24E−5 | 0.2066 | 9223 | 212 | 9.86E−7 |
| 2.81E−4 | 2.15E−5 | 0.1976 | 9191 | 244 | 1.152E−6 |
| 3.16E−4 | 2.11E−5 | 0.1940 | 9194 | 241 | 1.311E−6 |

From the graph and from the equation 1
1/Δε=0.0034, 1/Δε.Kₐₚₚ]=2 E-7
Therefore Kₐₚₚ.=0.0034/2E-7=1.70 E4 M
Δε=294
Therefore ε_{b}=ε_{f}−Δε=9435−294=9141

Titration of BISNMe₂ with DNA. 0.6 mg of BISNMe₂ was dissolved in 5 mL of DMSO. The resulting solution was diluted 5 times with 50 mM, pH 7, I=0.15 sodium phosphate buffer (physiological condition). The concentration of the diluted solution was 3.02 E-5 M. 2.3 mL of the diluted solution was pipetted into a UV-Visible cell and the absorption spectrum was taken. 0, 100 μL, 150 μL, 200 μL, 250 μL, 300 μL, 350 μL and 400 μL of DNA solutions were added, mixed well with a small disposable needle and spectra were recorded again. The concentration of the species, the apparent absorbance at 380 nm, the apparent extinction coefficients at 380 nm were determined from the absorptions spectra and tabulated in Table 3.

TABLE 3

| | | | | Δεₐₚₚ = | |
|---|---|---|---|---|---|
| [DNA] | [BISNMe₂] | A_{obs} | εₐₚₚ | ε_{f} − εₐₚₚ | [DNA]/Δεₐₚₚ |
| 0 | 3.02E−5 | 0.4550 | 15066 | 0 | 0 |
| 8.88E−5 | 2.89E−5 | 0.3419 | 11830 | 3236 | 2.74E−8 |
| 1.30E−4 | 2.84E−5 | 0.3086 | 10866 | 4200 | 3.10E−8 |
| 1.70E−4 | 2.78E−5 | 0.2896 | 10417 | 4649 | 3.66E−8 |
| 2.09E−4 | 2.72E−5 | 0.2756 | 10132 | 4934 | 4.24E−8 |
| 2.46E−4 | 2.67E−5 | 0.2668 | 9993 | 5073 | 4.85E−8 |
| 2.81E−4 | 2.62E−5 | 0.2644 | 10092 | 4974 | 5.65E−8 |
| 3.16E−4 | 2.57E−5 | 0.2526 | 9829 | 5237 | 6.03E−8 |

From the graph and from the equation 1
1/Δε=0.0002, 1/[Δε.Kₐₚₚ]=1 E-8
Therefore Kₐₚₚ.=0.0002/1 E-8=2.0 E4 M
Δε=5000
Therefore ε_{b}=ε_{f}−Δε=15066−5000=10066
Results and Discussion

Significant hypochromic effects, accompanied by change in the shape of the absorption bands, and fairly clear isosbestic points were observed in all three cases. These indicate the formation of ground state complexes of these Rh metal complexes with double helical ct-DNA. The isosbestic points suggest homogeneity of the Rh complex-ct-DNA binding (Salvatore, New J. Chem., 26:250-258 (2002)).

The intrinsic binding constant for OCTBP, BISTAP and BISNMe₂ are 4.25 E3 M, 1.70 E4 M and 2.0 E4 M respectively under physiological condition (ionic strength is 0.15). Compare to BISPHEN, which shows only a minimal association under very low ionic strength (100 M), these derivatives show greater association with DNA in the dark.

The association constants of the order of 10⁴ or greater is a typical to an intercalating drug. BISTAP has the extended planar aromatic ring, which helps the drug for a better stacking between the nucleic acid bases.

The greater association constant and a large bathochromic shift in BISNMe2 is somewhat interesting. The —NMe₂ group, when it is perpendicular to the “phen” ring can intercalate. It can also groove bind (hydrophobic interactions).

There is a report (Barton, et al., J. Am. Chem. Soc., 108:7414(1986)) that methylation of phenanthroline rings increases the groove binding of phenanthroline rings. The association in OCTBP, then, should be mainly groove binding. The relatively low association constant (4.25 E3) and hypochromic effect are in agreement with this prediction.

It should also be noted that in vitro kinetic and thermodynamic studies showed that OCTBP has greater affinity toward calf thymus DNA than BSA protein. The kinetic equilibrium results suggest that OCTBP can reach DNA even in the presence of large excess of protein.

### Example XII

### Reversed Phase HPLC Retention Times of Various Bisbipyridyl Rhodium (III) Complexes

Reversed phase high performance liquid chromatography (RP-HPLC) was carried out on C8 columns. Compounds were tested using 20%, 30% and 45% acetonitrile in 100 mM ammonium acetate at pH 5.4 for elution on a C8 column, with the intention of achieving an elution time of about 15 minutes. It was expected that the more hydrophobic a compound, the higher the acetonitrile content one would need for the eluent.

BISPHEN was found to elute at about 15 minutes in 20% acetonitrile; the tetramethyl analogs elute at about 15 minutes in 30% acetonitrile. Neither of these compounds is able to pass through a cell membrane. DPPZPHEN, BISNMe2 and OCTBP were found to require 45% acetonitrile to elute at around 15 minutes. These compounds are all able to pass through the cell membrane, and are phototoxic. BISTAP elutes earlier (8-10 minutes) in 45% acetonitrile, and does not get into the cells (it may not be hydrophobic enough). TMOBP and TIOBP (the tetraalkoxy compounds) elute at around 15 minutes in 45% acetonitrile. Based on their elution characteristics, it is expected that these compounds should get through the cell membrane and be active. The tetraphenyl compound (TPBP) and BISDPPZ elute at around 30 minutes in 45% acetonitrile and have not yet been tested for cell uptake or phototoxicity. Given the data obtained to date, it appears that elution of the bisbipyridyl rhodium (III) complex of the invention at about 15 minutes or more with 45% acetonitrile on a C8 column may be a good predictor of bioactivity.

### Example XIII

### Emission Properties of Various Bisbipyridyl Rhodium (III) Complexes

Phosphorescence spectra were obtained by using the SLM Aminco SPF-500 spectrophotometer using a 300 W xenon arc lamp operating in the A/B mode. Compounds examined included BISPHEN, methylated bisphens 37TMBP, 56TMBP and OCTBP, and 4,7-substituted bisphens TIOBP, TMOBP and BISNMe2. The emission intensities were corrected for the wavelength dependence of the detector sensitivity. The samples were in a methanol-water (4:1 by volume) glass using a standard quartz EPR tube in an optical dewar at 77K. The excitation wavelength was normally maintained at 355 nm or otherwise specified. Interference filters >380 nm and >385 nm were used to remove the frequency doubled and tripled interference in the emission. All the spectra were normalized to match with the emission spectra of BISPHEN.

Compared to BISPHEN, the emission maxima of methylated bisphens were blue shifted 15 to 20 nm the emission maxima. The blue shift is greater in 37TMBP and in OCTBP compared to 56TMBP. In general, all these emissions are broad and structureless.

The emitting state in all these cases is a metal-based triplet excited state. Since these states arise due to the electronic movement within the Rh metal atomic orbital they won't have the vibrational fine structure.

Methylation provides an increased electron density in the phenanthroline ligands, which makes them stronger ligands than the parent “phen” ligand. As a consequence the ligand field splitting Δ (10 Dq) will increase. In other words the energy gap between t_{2g} and e_{g} orbitals will increase. This should be reflected in the metal-based absorption and emission bands.

Since methyl groups mainly donate electron density through σ-bonds inductively, the effect will be minimal when they are far away from the metal as such in 56TMBP.

The very minimal change in OCTBP and 37TMBP hints that the “para” methylation has the most significant effect on the metal-based triplet excited state.

Compared to Rh (phen) Cl4⁻, which is also a known d-d emitter, methylation increases the energy of the lowest lying d-d excited state (Watts, JACS, 96:4334-4335 (1974)). In the case of Rh (phen) Cl4⁻ the energy of the d-d state is red shifted by methylation (the effect is smaller) whereas in our case it is blue shifted (the effect is comparatively larger). Watts argues that in Rh (phen) Cl4⁻ the σ-bonding increases are comparable to π-backbonding decreases due to methylation on the phenanthroline ligands. By analogy to Ir (phen) Cl4⁻, he also argues that, normally, π-backbonding decreases are far more important than any increases in σ-bonding. In our case, it seems that the reverse is true. This is further confirmed by blue shifts observed when iPro groups are on the phen ligands.

With respect to the 4,7-disubstituted BISPHENS, it was found that the isopropyl group in TIOBP causes a blue shift (12 nm) in the emission maxima, whereas dimethylamino group in BISNMe2 causes a considerable red shift (20 nm) in the maxima. O-methylation (TMOBP) does not seem to alter the emission maxima. Initial dual emissions from NMe₂ and isopropyl groups (second emission is structured and observed in the short wavelength region) were eliminated when triply recrystallized samples were used. In isopropyl groups, as in methyl groups, σ-bonding is more important than π-backbonding. They are balanced in methoxy groups, whereas in NMe₂, it seems the π-backbonding is more important than the σ-bonding.

It was found that methylation lowers the lowest lying triplet energy of the phenanthroline ligands, and further that the position of the methylation may be more important than that of the degree of methylation in lowering the triplet energy. For example, dimethylation at positions 5,6 lowers the triplet energy more compare to dimethylation at positions 3,7 and tetramethylation at positions 3,4,7,8. This order is reversed, in the ³(d-d) state energy of the bis Rh complexes of these ligands.

It was further found that the methoxy group does not alter the triplet energy significantly. The same trend is observed in bis Rh complexes of this ligand as well.

The NMe₂ substituent was found to lower the triplet energy, but the effect is comparable to that of 5,6-dimethylation. There is a small hump is observed in the lower energy region of the spectrum. It might be due to the dual phosphorescence observable in the polar media due to TICT phenomena.

The tap ligand (FIG. 2c) increases the low-lying triplet whereas dppz ligand (FIG. 2b) decreases the energy of the triplet-excited state. The reason for opposite effect of these two very similar compounds is not clear, since normally the extension of the conjugation should lower both singlet and triplet excited states energies.

### Example XIV

### Photoaquation of Methylated Cis-dichlorobis(1,10-phenanthroline) rhodium(III)chloride Compounds by Direct Population of a Photoactive Triplet Excited State

Cis-dichlorobis(1,10)-phenanthroline) rhodium (III) chloride, cis-Rh(phen)₂Cl₂⁺, and its analogs are chemically inert to aquation under thermal conditions in neutral aqueous media, but the chloride ligands are replaced sequentially by water upon UVA irradiation. In such bis(phenanthroline)Rh(III) complexes, excitation with UVA light typically populates low lying singlet ligand field states, the absorption for which occurs between 330 and 400 nm (DeArmond et al., J. Chem. Phys., 54:2247-2253 (1971)). Intersystem crossing to the triplet manifold from the initially generated excited singlet states is highly efficient in these rhodium complexes (Ford, Coord. Chem. Rev., 44:61 (1982); Ford et al., J. Prog. Inorg. Chem., 30:213 (1983); Skibsted, Coord. Chem. Rev., 64:343 (1985)) and their photoreactivity is generally ascribed to their ligand field triplet states.

We now report that one can generate analogous photochemistry in two methylated analogs of cis-Rh(phen)₂Cl₂⁺, 37TMBP and OCTBP, upon their direct excitation into the triplet manifold (i.e. with visible light >500 nm). To our knowledge, this is the first report of such ligand field photochemistry occurring by direct excitation into the triplet state of an octahedral rhodium(III) complex. It is also noteworthy that methyl substitution of the aromatic rings, as in 37TMBP and OCTBP, increases the triplet photoreactivity of these complexes by 10 fold relative to the unsubstituted analog, BISPHEN. We are unaware of any prior reports of such a dramatic effect of ring methylation of aryl ligands on the ligand field triplet photochemistry of low spin d⁶ transition metal complexes.

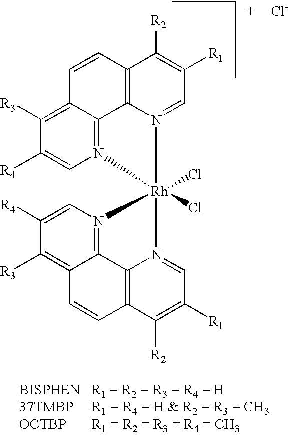

Aqueous solutions (3 mL, 0.1 mM) of the Rh complexes were irradiated for 8 hours under argon using a 450 W Hanovia medium pressure mercury lamp filtered through a uranium yellow glass (cut-off<330 nm) and a 1.5 cm 0.5% (w/v) aqueous solution of K₂Cr₂O₇ (0% transmittance≦500 nm). Photodestruction of the metal complexes was followed by HPLC analysis. Compound BISPHEN was virtually unaffected by such long-wavelength excitation but 37TMBP and OCTBP showed measurable levels of photodestruction (6.1 and 9.3% loss, respectively). The enhanced visible-light reactivity caused by methylation of the phenanthroline rings reflects the measured quantum efficiencies of photodestruction of these compounds at 311 nm (where precise extinction coefficients are known): OCTBP (0.20) 37TMBP (0.18)>>BISPHEN (0.02).

The major product of the long-wavelength photochemistry is the corresponding monoaquation product. This was confirmed by LC/MS-ESI analysis of the photolysate, which in each case provided a molecular ion for the major peak corresponding to the monoaquation product. ¹H-NMR spectral analyses of the reactions in D₂O are consistent with this assignment. The loss of symmetry upon replacement of a chloride by water in 37TMBP leads to a change from two to four singlets for the methyl resonances, and from one to two doublets for the downfield phenanthroline hydrogen closest to the chloride. Likewise, in the photolysis of OCTBP, the four methyl singlets double to eight and the single downfield phen-H singlet becomes two singlets.

To more specifically identify the transition responsible for the visible light photochemistry we determined an “action spectrum” for the photoaquation in compound OCTBP using dye lasers emitting between 520 and 610 nm. The percent photoaquation, normalized for photons incident on the laser cell at each wavelength indicates two maxima in the region of 540 and 580 nm. The transition at 580 nm matches well with the observed onset of phosphorescence emission at 77K from OCTBP. The phosphorescence onset for OCTBP at about 600 nm is consistent with a similar onset reported for BISPHEN (DeArmond, J. Chem. Phys., 54:2247-2253 (1971)). In the absence of vibronic fine structure one can use the onset of phosphorescence to approximate the energy of the 0,0 band of the singlet to triplet transition. Of course, our assumption here is but part of a set of arguments that include the action spectrum and the theoretical analysis. We were unsuccessful in observing uv-vis absorption bands for these complexes in this region using a 10 cm pathlength cell and a saturated aqueous solution (about 1.5 mM).

Time dependent density functional calculations (TD-DFT) (Casida, Recent Advances in Density Functional Theory Methods, Vol. 1, Chong, Ed.; World Scientific, Singapore (1995)) were carried out on BISPHEN and OCTBP. The lowest energy singlet to singlet excitations were calculated at 465 nm and 461 nm, both well to the blue of the wavelengths used for photochemical excitation in this study. The singlet to singlet excitations with largest oscillator strength were calculated at 256 nm and 291 nm, respectively. The calculated energies and intensities in the gas phase are in fair quantitative and qualitative agreement with the experimental absorption spectra recorded in water. Formally forbidden singlet to triplet excitations were also calculated for BISPHEN and OCTBP. Within the framework of TD-DFT all such excitations yield a zero oscillator strength and no absorption intensities are predicted. The lowest energy singlet to triplet excitations were calculated at 557 nm and 554 nm for BISPHEN and OCTBP, respectively. These latter values suggest that one might expect to initiate singlet to triplet excitation using wavelengths of light ≧about 550 nm.

One is left to explain why methylation of the phenanthroline rings enhances the photoaquation so markedly that reactivity is observed even upon direct singlet to triplet excitation. We do not believe that the answer lies in any significant rearrangement of metal and ligand centered excited states. Compound BISPHEN exhibits a low intensity (ε≦100) shoulder on the red edge (about 385 nm) of its UVA absorption that has been assigned to a ¹T₁←¹A₁ transition that populates a lowest-lying d-d state. Likewise, the lowest lying triplet for BISPHEN has been assigned as a d-d state based on the broad, structureless nature of its phosphorescence (λₘₐₓ at 710 nm), and the emission's insensitivity to solvent environment, its lack of vibrational fine structure and its relatively short life time (47.3 μs at 77K). In the methylated analogs, 37TMBP and OCTBP, the ligand absorption maxima are only slightly red-shifted (7-9 nm) relative to BISPHEN, and the d-d bands occur at wavelengths virtually identical to that in the parent complex. Both 37TMBP and OCTBP show phosphorescence with a maximum at about 710 nm (77K) that is Gaussian shaped and quite similar in appearance to the emission from BISPHEN. Others have observed that the d-d triplet state energy for K[Rh(phen)Cl₄] is also relatively insensitive to the presence of phenanthroline methyl substituents. (Watts, J. Am. Chem. Soc., 96:4334-4335 (1974)). We therefore assign the excited state directly populated by long-wavelength visible light to the metal centered ³T₁ state. Such transitions have been previously reported for trans-[Rh(en)₂Cl₂]Cl at about 469 nm (ε of about 1.5) and for trans-[Rh(py)₄Cl₂]Cl at about 465 nm (ε of about 3) as weak inflections in the low energy region of their absorption spectra (DeArmond et al., J. Chem. Phys., 54:2247-2253 (1971)).

If the state assignment for the lowest lying triplet remains unaltered by methyl substitution on the phenanthroline rings, why then is visible light-initiated photochemistry observed for compounds 37TMBP and OCTBP and not for BISPHEN? It is likely that the extinction coefficients for the singlet-triplet transitions in this series are not markedly affected by the methylation since the extinction coefficients for the singlet-singlet ligand field transitions in all these complexes are virtually the same. We believe the answer lies in the 10-fold greater quantum efficiencies for photoaquation observed at 311 nm. Since intersystem crossing is likely to be virtually complete in all three rhodium complexes the enhanced quantum efficiencies of the methylated analogs may be due to an increased rate of aquation of their metal-centered triplet states. A dissociation/addition mechanism is generally accepted as responsible for the substitution chemistry associated with a hexacoordinated Rh(III) ligand field triplet (Weiland et al., Inorg. Chem., 25:4893 (1986)). The dissociation step is due to the promotion of an electron from the t_{2g} orbital to the e_{g} orbital, which leads to significant weakening of the metal ligand bonds. (Hipps et al., Inorg. Chem., 13:1544 (1974)). The increased reactivity exhibited by 37TMBP and OCTBP may be attributed to an increase in σ-donation from the methylated phen ligands to the Rh (Ford, Rev. Chem. Intermed., 2:267-296 (1978)), which should markedly stabilize the pentacoordinate species formed by the dissociation of the labile Cl⁻ and/or the transition state leading to it. In fact, there is evidence that the reactivity of 37TMBP and OCTBP is enhanced in the ground state in a similar fashion. Though all of these complexes are extremely stable to dark thermal aquation, they can be hydrolyzed by base, and we have observed that such thermal hydrolysis at 80° C. with 10% NaOH follows the same trend as that of photoaquation, i.e. OCTBP≅37TMBP>BISPHEN.

The complete disclosures of all patents, patent applications including provisional patent applications, and publications, and electronically available material (e.g., GenBank amino acid and nucleotide sequence submissions) cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

1. A compound having formula I 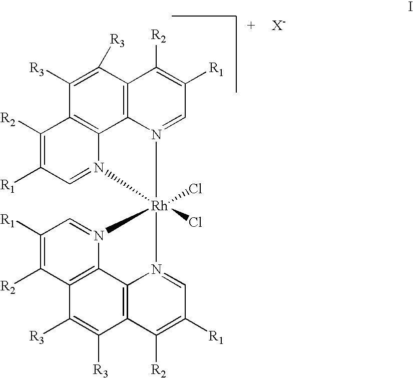
wherein R₁, R₂ and R₃ are each independently selected from the group consisting of an alkyl group, an alkenyl group, an alkynyl group, a nitrile, an azide, an aryl group, an aralkyl group, a heteroaryl group, a hydroxy group, an alkoxy group, an aryloxy group, an amine group, and a hydrogen atom, or any two of R₁, R₂ and R₃ together form an aryl or heteroaryl ring; and wherein X is a counterion, with the proviso that where R₁=R₃=H, R₂ is neither methyl nor phenyl.

2. A compound having the formula II 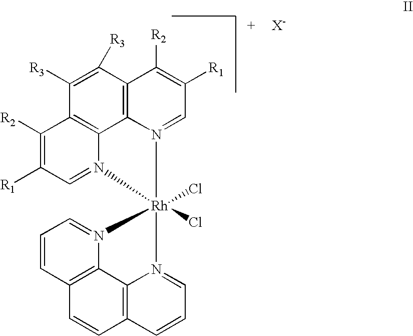
wherein R₁, R₂ and R₃ are each independently selected from the group consisting of an alkyl group, an alkenyl group, an alkynyl group, a nitrile, an azide, an aryl group, an aralkyl group, a heteroaryl group, a hydroxy group, an alkoxy group, an aryloxy group, an amine group, and a hydrogen atom, or any two of R₁, R₂ and R₃ together form an aryl or heteroaryl ring, and wherein X is a counterion.

3. The compound of claim 1 or 2 wherein R₁=R₂=H and R₃=(C1-C4) alkyl.

4. The compound of claim 1 wherein R₁=R₂=H and R₃=CH₃ (cis-dichlorobis(5,6-dimethyl-1,10-phenanthroline)rhodium (III) chloride; 56TMBP).

5. The compound of claim 1 or 2 wherein R₁=R₂=(C1-C4) alkyl and R₃=H.

6. The compound of claim 1 wherein R₁=R₂=CH₃ and R₃=H (cis-dishlorobis(3,4,7,8-tetramethyl-1,10-phenanthroline)rhodium(III)chloride; OCTBP)

7. The compound of claim 1 or 2 wherein R₁=R₃=H and R₂=N—(C1-C4)alkyl.

8. The compound of claim 1 wherein R₁=R₃=H and R₂=N(CH₃)₂ (cis-dichlorobis{N,N-dimethylamino)-1,10-phenanthroline}rhodium(III)chloride; BISNMe2).

9. The compound of claim 1 or 2 wherein R₁=R₃=H and R₂=O—(C1-C4)alkyl.

10. The compound of claim 1 wherein R₁=R₃=H and R₂=O—CH₃ (cis-dichlorobis(3,7-dimethoxy-1,10-phenanthroline)rhodium(III)chloride; TMOBP).

11. The compound of claim 1 wherein R₁=R₃=H and R₂=O—(CH₂)(CH₃)₂ (cis-dichlorobis(3,7-diisopropoxy-1,10-phenanthroline)rhodium(III)chloride; TIOBP.

12. The compound of claim 1 or 2 wherein R₁=R₃=H and R₂=(C1-C4)phenyl.

13. Cis-dichloro{2,3-di(2-pyridyl)quinoxaline}{1,10-phenanthroline}rhodium (III) chloride (TAPPHEN).

14. Cis-dichlorobis{2,3-di(2-pyridyl)quinoxaline} rhodium (III) chloride (BISTAP).

15. Cis-dichloro(dipyrido[3,2a-2′3′c]phenazine)(1,10-phenantroline) rhodium (III) chloride (DPPZPHEN).

16. Cis-dichlorobis {dipyrido(3,2-a: 2′,3′-c)phenazine}rhodium (III) chloride (BISDPPZ).

17. A method for reducing the level of pathogenic contaminants in a biological material comprising:
(a) contacting the biological material with an effective amount of at least one bisbipyridyl rhodium (III) compound having the formula I, II, III or IV: 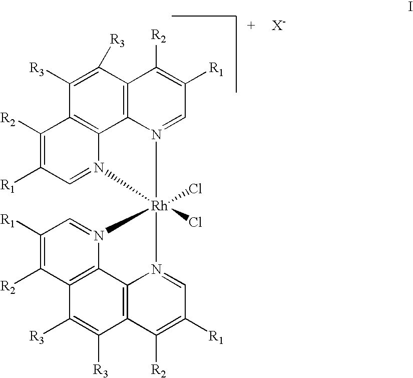
wherein R₁, R₂ and R₃ are each independently selected from the group consisting of an alkyl group, an alkenyl group, an alkynyl group, a nitrile, an azide, an aryl group, an aralkyl group, a heteroaryl group, a hydroxy group, an alkoxy group, an aryl oxy group, an amine group, and a hydrogen atom, or any two of R₁, R₂ and R₃ together form an aryl or heteroaryl ring; and wherein X is a counterion, with the proviso that where R₁=R₃=H, R₂ is not methyl, and where R₁=R₂=H, R₃ is not methyl, 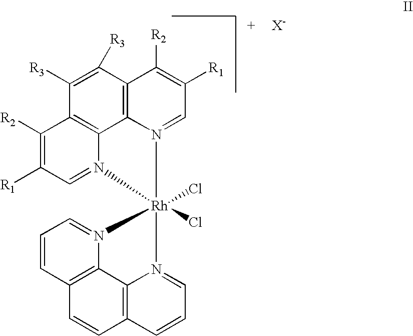
wherein R₁, R₂ and R₃ are each independently selected from the group consisting of an alkyl group, an alkenyl group, an alkynyl group, a nitrile, an azide, an aryl group, an aralkyl group, a heteroaryl group, a hydroxy group, an alkoxy group, an aryloxy group, an amine group, and a hydrogen atom, or any two of R₁, R₂ and R₃ together form an aryl or heteroaryl ring, and wherein X is a counterion; 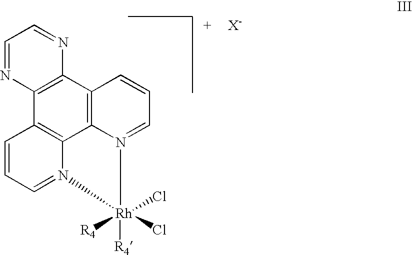
wherein R₄ and R₄′ together form a phen ligand, yielding cis-dichloro{2,3-di(2-pyridyl)quinoxaline}{1,10-phenanthroline}rhodium (III)chloride (TAPPHEN) or a “tap” ligand, yielding cis-dichlorobis{2,3-di(2-pyridyl)quinoxaline} rhodium (III) chloride (BISTAP); 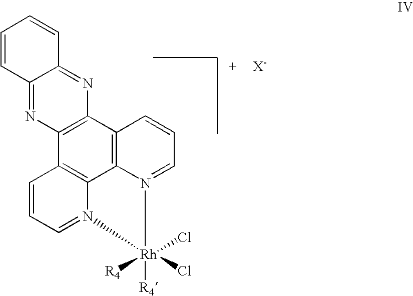
wherein R₄ and R₄′ together form a phen ligand, yielding cis-dichloro(dipyrido[3,2a-2′3′c]phenazine)(1,10-phenantroline) rhodium(III) chloride (DPPZPHEN) or a dppz ligand, yielding cis-dichlorobis{dipyrido(3,2-a: 2′,3′-c)phenazine}rhodium (III) chloride (BISDPPZ); and
(b) irradiating said biological material for a time sufficient to activate the bisbipyridyl rhodium (III) compound thereby causing a reduction the level of said pathogenic contaminants in said biological material.

18. The method of claim 17 wherein the biological material comprises blood, semen, ascites fluid, milk, lymphatic fluid, an organ, a tissue, a hybridoma cell line, or components thereof.

19. The method of claim 17 wherein the biological material comprises blood or blood components.

20. The method of claim 19 wherein the biological material is substantially free of hemoglobin.

21. The method of claim 20 wherein the biological material comprises at least one blood component selected from the group consisting of platelets, concentrated platelets, plasma, serum and blood proteins.

22. The method of claim 17 wherein the biological material comprise diseased cells in a patient.

23. The method of claim 22 wherein the diseased cells are tumor cells.

24. The method of claim 17 further comprising removing the biological material from patient prior to contacting the biological material with the bisbipyridyl rhodium (III) compound.

25. The method of claim 24 wherein the biological material comprises blood, blood components, or tumor cells.

26. The method of claim 24 further comprising returning the biological material to the patent after irradiation.

27. The method of claim 17 wherein the biological material comprises tumor cells, the method further comprising returning the tumor cells to the patient prior to irradiation.

28. The method of claim 17 wherein step (b) comprises irradiating the biological material with light having a wavelength of 310 nm to 400 nm.

29. The method of claim 28 wherein the irradiating light has a wavelength of 320 nm to 400 nm.

30. The method of claim 17 wherein step (b) comprises irradiating the biological material with light having a wavelength of >400 nm.

31. The method of claim 17 wherein the pathogenic contaminant comprises a pathogenic organism selected from the group consisting of a bacterium, virus and protozoan.

32. The method of claim 17 wherein the pathogenic contaminant comprises a leukocyte.

33. The method of claim 17 wherein the pathogenic contaminant comprises a tumor cell.

34. The method of claim 17 further comprising, after step (b), removing the bisbipyridyl rhodium (III) compound from the biological material.

35. The method of claim 17 further comprising, prior to step (b), contacting the biological material with a sensitizer molecule having an absorption maximum of greater than 550 nm; wherein step (b) comprises irradiating the biological material with light having a wavelength of greater than 550 nm so as to excite the sensitizer molecule and thereby indirectly activate the bisbipyridyl rhodium (III) compound.

36. The method of claim 35 wherein the biological material comprises blood or blood components.

37. The method of claim 35 wherein the biological material comprises red blood cells.

38. The method of claim 35 wherein the biological material comprises hemoglobin.

39. The method of claim 35 wherein the irradiation sensitizer molecule comprises methylene blue or a derivative thereof.

40. The method of claim 35 wherein irradiation sensitizer molecule comprises acridine orange or a derivative thereof.

41. The method of claim 17 or 35 wherein the bisbipyridyl rhodium (III) compound is selected from the group consisting of cis-dichloro(dipyrido[3,2a-2′3′c]phenazine)(1,10-phenantroline) rhodium(III) chloride (DPPZPHEN), cis-dichlorobis(3,4,7,8-tetramethyl-1,10-phenanthroline) rhodium(III) chloride (OCTMP), cis-dichlorobis{dipyrido(3,2-a: 2′,3′-c)phenazine}rhodium (III) chloride (BISDPPZ), cis-dichlorobis(3,7-dimethoxy-1,10-phenanthroline) rhodium(III) chloride (TMOBP), cis-dichlorobis(3,7-diisopropoxy-1,10-phenanthroline) rhodium(III) chloride (TIOBP), cis-dichlorobis{3,7(N,N-dimethylamino)-1,10-phenanthroline} rhodium(III) chloride (BISNMe2), cis-dichlorobis(4,7-diphenyl-1,10-phenanthroline) rhodium(III) chloride (TPBP), and cis-dichlorobis{2,3-di(2-pyridyl)quinoxaline} rhodium (III) chloride (BISTAP).

42. The method of claim 17 or 35 wherein the bisbipyridyl rhodium (III) compound is DPPZPHEN, OCTMP or BISNMe2
